(19) United States
(12) Patent Application Publication
(10) Pub. No.: US 2015/0228907 A1
(43) Pub. Date: Aug. 13, 2015

(54) Porous Films for Use in Light-Emitting Devices

(71) Applicant: Nitto Denko Corporation, Osaka (JP)

(72) Inventor: Liping Ma, San Diego, CA (US)

(73) Assignee: Nitto Denko Corporation, Osaka (JP)

(21) Appl. No.: 14/424,302

(22) PCT Filed: Aug. 29, 2013
(86) PCT No.: PCT/US2013/057364

§ 371 Date: Feb. 26, 2015

Related U.S. Application Data
(60) Provisional application No. 61/696,085, filed on Aug. 31, 2012.

(51) Int. Cl. H01L 51/00; C07D 413/14

(52) U.S. Cl. CPC H01L 51/0061; C07D 413/14; H01L 51/0067; H01L 51/0071; H01L 51/5271

(57) ABSTRACT

Some porous films, such as organic non-polymeric porous films, may be useful for light outcoupling to increase light-emitting device efficiency. They may also be used for light scattering in other devices and for other applications related to the transfer of light.

### SUMMARY

Some embodiments relate to porous films, such as porous films for use in devices, such as light-emitting devices.

Some embodiments may include a light-emitting device comprising: a light-emitting diode comprising a porous film; wherein the porous film may be disposed on an internally reflective layer, wherein the internally reflective layer is: an anode; a cathode; a transparent layer disposed between the anode and the porous film, or a transparent layer disposed between the cathode and the porous film.

These and other embodiments are described in detail herein.

### BRIEF DESCRIPTION OF THE DRAWINGS

FIG. 1 is a depicted to provide assistance in determining an x dimension, a y dimension, and a z dimension of a particle or protrusion.

FIG. 2A depicts an idealized example of a particle that may be described as: substantially rectangular when viewed in the xz plane and pseudoplanar.

FIG. 2B depicts an example of a particle that may be described as a curved or wavy nanoflake.

FIG. 3 depicts an idealized example of a particle having substantially all substantially right angles in the plane.

FIG. 4 is an idealized example of a pseudo-paralellogramatic particle having angles that may not be substantially right angles.

FIG. 5 depicts an idealized example of a substantially capsule-shaped particle.

FIG. 6 depicts an SEM image of a surface of a porous film.

FIG. 7 depicts an SEM image of a surface of a porous film.

FIG. 8 depicts an SEM image of a surface of a porous film.

FIG. 9 depicts an SEM image of a surface of a porous film.

FIG. 10 depicts an SEM image of a surface of a porous film.

FIG. 11 depicts an SEM image of a surface of a porous film.

FIG. 12 depicts an SEM image of a surface of a porous film.

FIG. 13 depicts an SEM image of a surface of a porous film.

FIG. 14 depicts an SEM image of a surface of a porous film.

FIG. 15 depicts an SEM image of a surface of a porous film.

FIG. 16 depicts an SEM image of a surface of a porous film.

FIG. 17 depicts an SEM image of a surface of a porous film.

FIG. 18 depicts an SEM image of a surface of a porous film.

FIG. 19 depicts an SEM image of a surface of a porous film.

FIG. 20 depicts an SEM image of a surface of a porous film.

FIG. 21 depicts an SEM image of a surface of a porous film.

FIG. 22 depicts an SEM image of a surface of a porous film.

FIG. 23 depicts an SEM image of a surface of a porous film.

FIG. 24 depicts an SEM image of a surface of a porous film.

FIG. 25 depicts an SEM image of a surface of a porous film.

FIG. 26 depicts an SEM image of a surface of a porous film.

FIG. 27 depicts an SEM image of a surface of a porous film.

FIG. 28 depicts an SEM image of a surface of a porous film.

FIG. 29 depicts an SEM image of a surface of a porous film.

FIG. 30 depicts an SEM image of a surface of a porous film.

FIG. 31 depicts an SEM image of a surface of a porous film.

FIG. 32 depicts an SEM image of a surface of a porous film.

FIG. 33 depicts an SEM image of a surface of a porous film.

FIG. 34 depicts an SEM image of a surface of a porous film.

FIG. 35 depicts an SEM image of a surface of a porous film.

FIG. 36 depicts an SEM image of a surface of a porous film.

FIG. 37 depicts an SEM image of a surface of a porous film.

FIG. 38 depicts an SEM image of a surface of a porous film.

FIG. 39 depicts an SEM image of a surface of a porous film.

FIG. 40 depicts an SEM image of a surface of a porous film.

FIG. 41 depicts an SEM image of a surface of a porous film.

FIG. 42 depicts an SEM image of a surface of a porous film.

FIG. 43 depicts an SEM image of a surface of a porous film.

FIG. 44 depicts an SEM image of a surface of a porous film.

FIG. 45 depicts an SEM image of a surface of a porous film.

FIG. 46 depicts an SEM image of a surface of a porous film.

FIG. 47 depicts an SEM image of a surface of a porous film.

FIG. 48 depicts an SEM image of a surface of a porous film.

FIG. 49 depicts an SEM image of a surface of a porous film.

FIG. 50 depicts an SEM image of a surface of a porous film.

FIG. 51 depicts an SEM image of a surface of a porous film.

FIG. 52 depicts an SEM image of a surface of a porous film.

FIG. 53 depicts an SEM image of a surface of a porous film.

FIG. 54 depicts an SEM image of a surface of a porous film.

FIG. 55 is a schematic diagram of some embodiments of a device described herein.

FIG. 56 is a schematic diagram of some embodiments of a device described herein.

FIG. 57 is a schematic diagram of some embodiments of a device described herein.

FIG. 58 is a schematic diagram of some embodiments of a device described herein.

FIG. 59 is a schematic diagram of some embodiments a device described herein.

FIG. 60 is a schematic diagram of some embodiments of a device described herein.

FIG. 61 is a schematic diagram related to preparation of a device described herein.

FIG. 62A is a schematic diagram of some embodiments a device described herein.

FIG. 62B is a schematic diagram related to preparation of a device described herein.

### DETAILED DESCRIPTION

Organic light-emitting devices (OLED) may be useful for incorporating into energy-efficient lighting equipment or devices. Unfortunately, the efficiency of OLEDs may be limited by both any inherent inefficiency in producing emitted light, and in the ability of emitted light to escape the device to provide lighting. The inability of emitted light to escape the device may also be referred to as trapping. Because of trapping, the efficiency of a device may be reduced to about 10-30% of the emissive efficiency. Light extraction may reduce trapping and thus substantially improve efficiency.

The porous films described herein may be useful in a variety of devices involving the transmission of light from one layer to another, such as light-emitting diodes, photovoltaics, detectors, etc. In some embodiments, a porous film may provide efficient light outcoupling for organic light-emitting diodes for uses such as lighting. With some devices, light extraction from a substrate close to 90%, or possibly greater, may be achieved. The porous films may provide easy processing and potentially low cost improvement in device efficiency.

In some embodiments, the porous films described herein may improve efficiency of a device by reducing the amount of total internal reflection in a layer of the device. Total internal reflection may be a significant cause of trapping. When light passes from a high refractive index material to a low refractive index material, the light may be bent in a direction away from the normal angle to the interface. If light in a higher refractive index material encounters an interface with a lower refractive index material at an angle which deviates substantially from 90°, the bending of the light may be greater than the angle at which the light approaches the interface, so that instead of passing out of the higher refractive index material, the light may be bent back into the higher refractive index material. This may be referred to as total internal reflection. Since air may have a lower refractive index than many materials, many interfaces between a device and air may suffer from loss due to total internal reflection. Furthermore, trapping due to total internal reflection may occur at any interface in a device where the light travels from a higher refractive index layer to a lower refractive index layer. Devices comprising porous films describe herein may have reduced total internal reflection or trapping and thus have improved efficiency. In some embodiments, a porous film disposed on an internally reflective surface may reduce total internal reflection by at least about 10%, at least about 20%, at least about 50%, at least about 70%, at least about 80%, at least about 90%, up to nearly 100%.

In some embodiments, a porous film described herein may provide light scattering for a variety of devices that involve light passing from one material to another, including devices that absorb or emit light. Light scattering may be useful in a device to provide viewing angle color consistency, so that the color is substantially similar regardless of the angle from which light is viewed. Devices having no light scattering layer may emit light in such a way that the viewer observes a different color depending upon the angle from which the light is viewed.

In some embodiments, a porous film described herein may also be useful as a filter for a variety of devices that involve light passing from one material to another, including devices that absorb or emit light

A porous film may include any film comprising a plurality of pores. For example, a porous film may comprise an irregularly oriented intermeshed nanostructure. Additionally, a porous film need not be a complete film or layer. For example, a porous film could include a plurality of nanoparticles, microparticles, nanostructures, or microstructures, that are dispersed on, but do not necessarily cover, the surface upon which the porous film is deposited.

In some embodiments a porous film may be deposited on a transparent substrate, which may reduce the total internal reflection of light within the substrate.

In some embodiments, a porous film may comprise a first surface and a second surface, wherein the first surface has a coplanar area that is substantially greater than a coplanar area of the second surface. While “coplanar area” is a broad term, one way to determine the coplanar area of a surface may be to place the surface under consideration on a smooth flat surface, and measure the area of the surface that contacts the smooth flat surface.

A porous film may have a variety of structures. In some embodiments, a porous film may have a surface comprising a plurality of irregularly arranged protrusions, particles, or aggregates thereof. The protrusions or particles may be nanoprotrusions, including nanoprotrusions having one or more dimensions in the nanometer to micron range. For example, nanoprotrusions or nanoparticles may have: an average x dimension of about 400 nm, about 500 nm, about 1000 nm, about 1500 nm, about 2000 nm, about 2500 nm, about 3000 nm, or any value in a range bounded by, or between, any of these lengths; an average y dimension of about 50 nm, about 100 nm, about 300 nm, about 500 nm, about 700 nm, about 1000 nm, about 1200 nm, about 1500 nm, about 1800 nm, about 2000 nm, or any value in a range bounded by, or between, any of these lengths; and/or an average z dimension of about 10 nm, about 30 nm, about 50 nm, about 70 nm, about 90 nm, about 100 nm, or any value in a range bounded by, or between, any of these lengths. In some embodiments, at least one particle in the film, or average of the particles in the film, may have an x dimension, a y dimension, or a z dimension of: about 5 nm, about 0.01 μm, about 0.02 μm, about 0.05 μm, about 0.1 μm, about 0.5 μm, about 1 μm, about 2 μm, about 5 μm, about 10 μm, about 20 μm, about 50 μm, about 100 μm, about 150 μm, about 200 μm, about 500 μm, about 1000 μm, or any length in a range bounded by, or between, any of these values.

In some embodiments, the protrusions, particles, or aggregates thereof may be substantially transparent or substantially translucent.

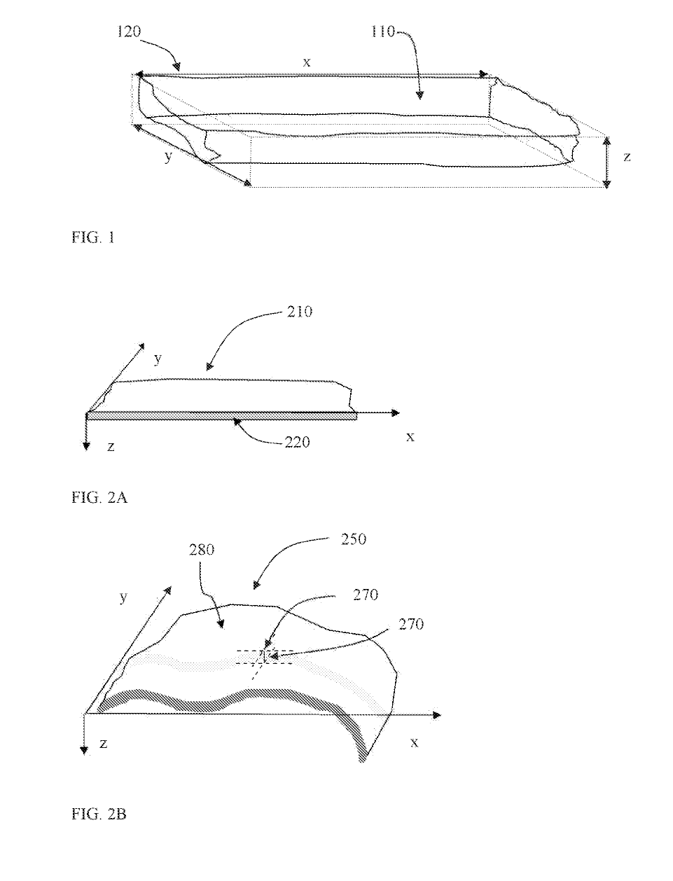

Although the particles, protrusions, or voids may be irregularly shaped, three dimensions, x, y, and z, may be quantified as depicted in FIG. 1. If a box 120 the shape of a rectangular prism is formed around the particle 110, or an open box the shape of a rectangular prism is formed around the protrusion, so that the box is as small as possible while still having the particle (or as much of protrusion as possible without altering the dimensions of the open end of the box) contained in it, the x dimension is the longest dimension of the box, the y dimension is the second longest dimension of the box, and the z dimension is the third longest dimension of the box.

The three dimensional shapes of the particles or protrusions may be characterized by describing the shape of the particles or protrusions when viewed in a certain plane. For example, a particle or protrusion may be substantially rectangular, substantially square, substantially elliptical, substantially circular, substantially triagonal, substantially parallelogramatic, etc., when viewed in the two dimensions of the xy, xz, or yz plane. The particular shape need not be geometrically perfect, but need only be recognizable as reasonably similar to a known shape. The three dimensional shape of the particles or protrusions might also be characterized or described using other terms.

FIG. 2A depicts an idealized example of a particle 210 that is substantially rectangular 220 when viewed in the xz plane. As depicted in this figure, the particle appears perfectly rectangular, but the shape need only be recognizable as similar to a rectangle to be substantially rectangular when viewed in the xz plane or any other plane.

With respect to FIG. 2A, the particle 210 may also be described as substantially linear when viewed in the xz plane because the x dimension is much greater than the z dimension. As depicted in this figure, the particle appears perfectly straight in the x dimension, but the shape need only be recognizable as similar to a line to be substantially linear when viewed in the xz plane or any other plane.

The particle 210 may also be described as a nanoflake. The term “nanoflake” is a broad term that includes particles that are flake-like in shape and have any dimension in the nanometer to micrometer range. This may include particles that are relatively thin in one dimension (e.g. z) and have a relatively large area in another two dimensions (e.g. xy).

The larger area surface need only be identifiable, but does not need to be planar. For example, the larger area surface may be substantially in the xy plane, such as particle 210, but may also be curved or wavy, such that substantial portions of the surface are not in the plane.

The particle 210 may also be described as pseudoplanar. The term “pseudoplanar” is a broad term that includes particles that are essentially planar. For example, a pseudoplanar particle may have a z dimension that is relatively insignificant as compared to the xy area of the particle that is substantially in the xy plane.

In FIG. 2B particle 250 is an example of a curved or wavy nanoflake. If substantial portions of the surface are not in the plane, a nanoflake may include particles having a large curved or wavy surface 260 and a small thickness 270 normal to a given point 280 on the surface.

With respect to any nanoflake or pseudoplanar particle or protrusion, including particle 210, particle 250, and the like, the ratio of the square root of the larger area or surface to a smallest dimension or a thickness normal to a point on the large surface (such as the ratio of the square root of an xy area to a z dimension), may be: about 3, about 5, about 10, about 20, about 100, about 1000, about 10,000, about 100,000, or any value in a range bounded by, or between, any of these ratios.

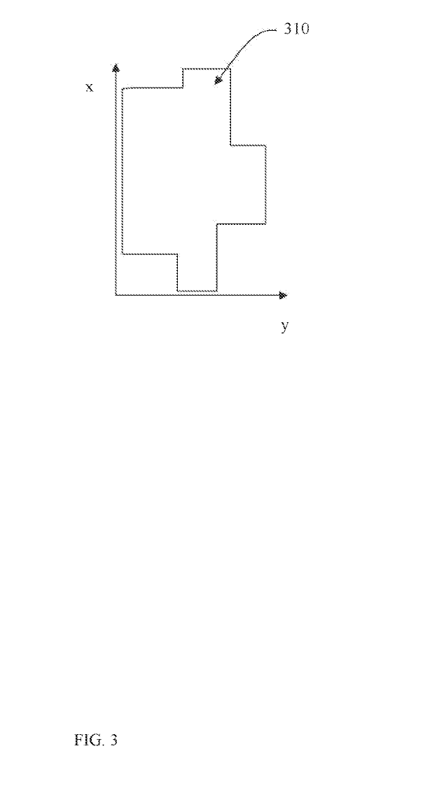

FIG. 3 depicts an idealized example of a particle 310 having substantially all substantially right angles in the xy plane. While not depicted in this figure, some particles may not have substantially all substantially right angles, but may have at least one substantially right angle. The particle 310 of this figure may also be described as pseudo-parallelogramatic. A pseudo-parallelogramatic particle may include two substantially linear portions of outer edges the particle that are substantially parallel viewed in the two dimensions of the xy, xz, or yz plane.

The outer edges of the particle may consist essentially of a plurality of linear edge portions.

Pseudo-parallelogramatic particles may have substantially right angles such as those depicted in FIG. 3, or they may have angles that may not be substantially right angles.

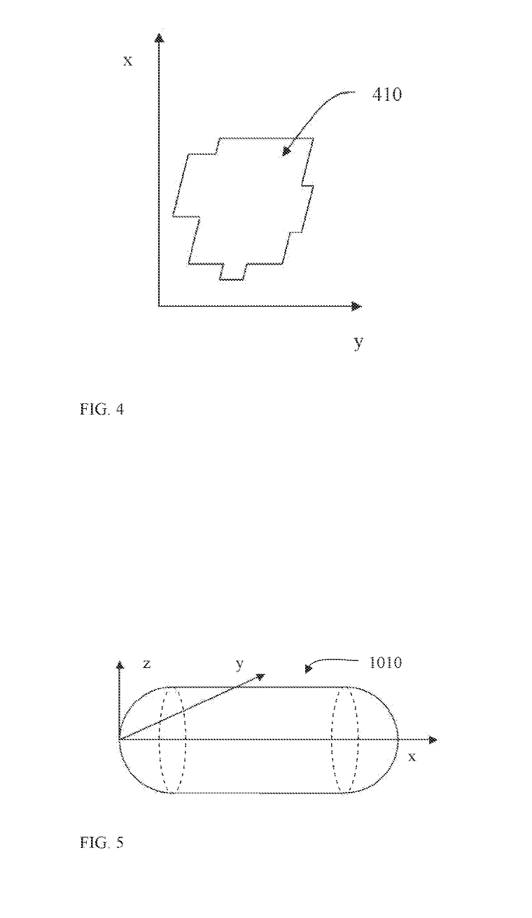

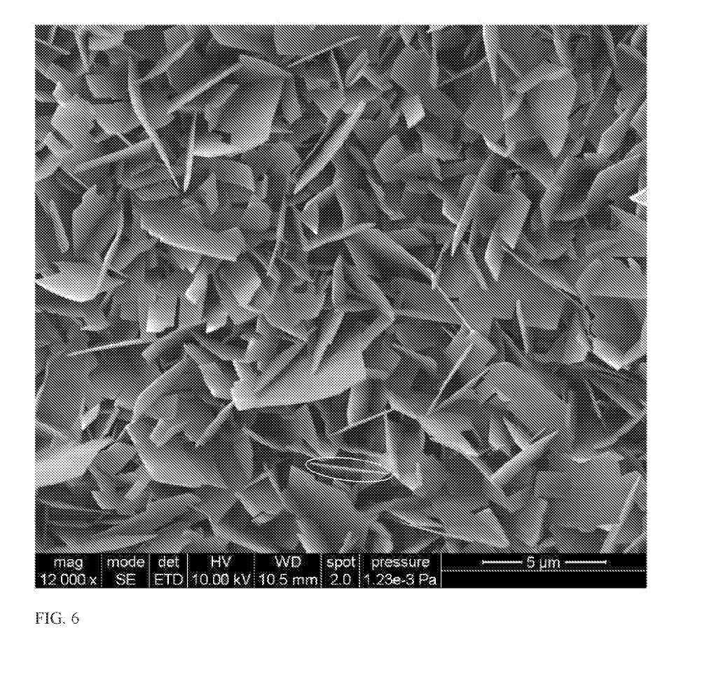

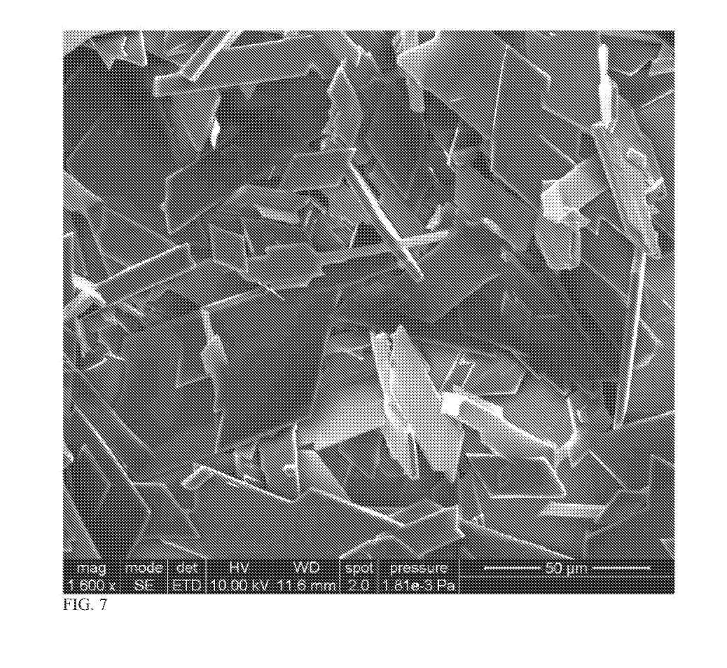

FIG. 4 is an idealized example of a pseudo-paralellogramatic particle 410 having angles that may not be substantially right angles.

A particle or protrusion may be described as needlelike if it has a shape that is reasonably recognizable as similar to a shape of a needle.

A particle or protrusion may be described as fiber-shaped if it has a shape that is reasonably recognizable as similar to a shape of a fiber.

A particle or protrusion may be described as ribbon-shaped if it has a shape that is reasonably recognizable as similar to the shape of a ribbon. This may include particles or protrusions that have a flat rectangular surface that is elongated in one dimension and thin in another dimension. The ribbon shape may also be curved or twisted, so that the particle need not be substantially coplanar to be ribbon-shaped.

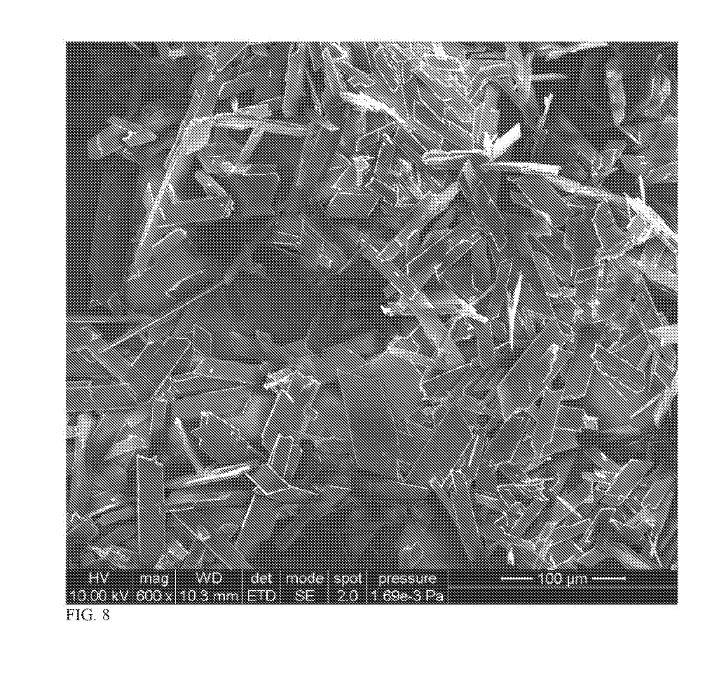

FIG. 5 depicts an idealized example of a substantially capsule-shaped particle 1010. When viewed in the xy or the xz plane, the particle 1010 may also be described as substantially oval. When viewed in the yz plane, the particle 1010 may also be described as substantially circular.

A particle or protrusion may be described as rod-shaped if it has a shape that is reasonably recognizable as similar to the shape of a rod. This may include particles or protrusions that are elongated in one dimension. A rod-shaped particle or protrusion may be substantially straight, or have some curvature or bending.

A particle or protrusion may be described as granular if the x, y, and z dimensions are similar, such as within an order of magnitude of one another.

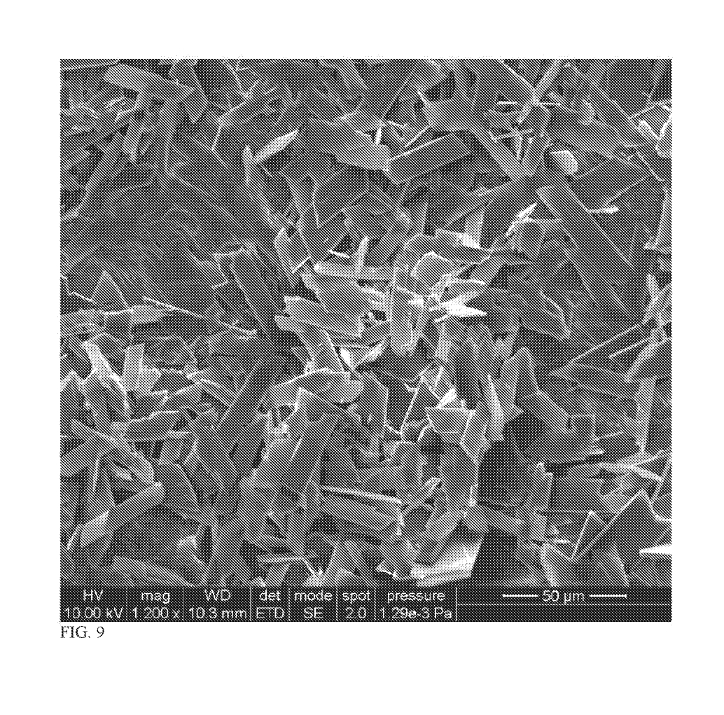

FIGS. 6-53 depict SEM images of actual porous films. All SEM images were recorded using a FEI xTm “Inspect F” SEM; 2007 model, version 3.3.2. In these figures, “mag” indicates the magnification level of the image, “mode” indicates the type of detector used to generate the image, where “SE” stands for secondary electron mode, “HV” indicates the accelerating voltage of the electron beam used to generate the image “WD” indicates the working distance between the detector and the actual surface being imaged, “spot” indicates a unitless indicator of the electron beam diameter, and “pressure” indicates the pressure, in pascals, within the microscope chamber at the time of image capture.

FIG. 6 depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: pseudo-parallelogramatic, at least one substantially right angle, and substantially all substantially right angles. Although not exhaustive, the following descriptions may apply to at least one of the protrusions or particles in this figure when viewed in the yz plane: substantially rectangular, substantially linear, and substantially all substantially right angles. Although not exhaustive, the following other descriptions may also apply to at least one of the protrusions or particles in this figure: nanoflakes and pseudoplanar.

A scale bar of 5 μm is indicated in the SEM, which may provide an indication of the size of the nanoparticles, nanoprotrusions, or voids of the film. This figure shows that a substantial number of particles or voids may have an x, y, and/or z dimension in the range of about 1 μm to about 20 μm. A substantial number of particles may also have a ratio of the square root of the xy area to the z dimension in the range of about 10 to about 100. For example, the particle circled in the figure appears to have a ratio:

$$\frac{{[ xy area ]}^{1 / 2}}{z}$$

of about 40, assuming that the length of the visible edge is about equal to the square root of the area. This method may be used for films such as the one depicted here, where, based upon other nanoflakes visible in the figure, the large area, or the xy area, is about equal to the length of one side viewed in the yz plane. Moreover, at least about 50%, about 70%, or about 90% of the particles on the surface may have a ratio of the square root of the xy area to the z dimension in the range of about 10 to about 1000.

FIG. 7 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: pseudo-parallelogramatic and substantially parallelogramatic. Although not exhaustive, the following descriptions may apply to at least one of the protrusions or particles in this figure when viewed in the yz plane: substantially rectangular, substantially linear, and substantially all substantially right angles. Although not exhaustive, the following other descriptions may also apply to at least one of the protrusions or particles in this figure: nanoflakes and pseudoplanar.

A scale bar of 50 μm is indicated in the SEM of FIG. 7, which may provide an indication of the size of the nanoparticles, nanoprotrusions, or voids of the film. This figure shows that a substantial number of particles or voids may have an x, y, and/or z dimension in the range of about 1 μm to about 500 μm. A substantial number of particles may also have a ratio of the square root of the xy area to the z dimension in the range of about 5 to about 100.

FIG. 8 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: pseudo-parallelogramatic and substantially parallelogramatic. Although not exhaustive, the following descriptions may apply to at least one of the protrusions or particles in this figure when viewed in the yz plane: substantially rectangular, substantially linear, at least one substantially right angle, and substantially all substantially right angles. Although not exhaustive, the following other descriptions may also apply to at least one of the protrusions or particles in this figure: nanoflakes and pseudoplanar.

A scale bar of 100 μm is indicated in the SEM of FIG. 8, which may provide an indication of the size of the nanoparticles, nanoprotrusions, or voids of the film. This figure shows that a substantial number of particles or voids may have an x, y, and/or z dimension in the range of about 1 μm to about 500 μm. A substantial number of particles may also have a ratio of the square root of the xy area to the z dimension in the range of about 5 to about 100.

FIG. 9 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: pseudo-parallelogramatic and substantially parallelogramatic. Although not exhaustive, the following descriptions may apply to at least one of the protrusions or particles in this figure when viewed in the yz plane: substantially linear. Although not exhaustive, the following other descriptions may also apply to at least one of the protrusions or particles in this figure: nanoflakes and pseudoplanar.

A scale bar of 50 μm is indicated in the SEM of FIG. 9, which may provide an indication of the size of the nanoparticles, nanoprotrusions, or voids of the film. This figure shows that a substantial number of particles or voids may have an x, y, and/or z dimension in the range of about 1 μm to about 500 μm.

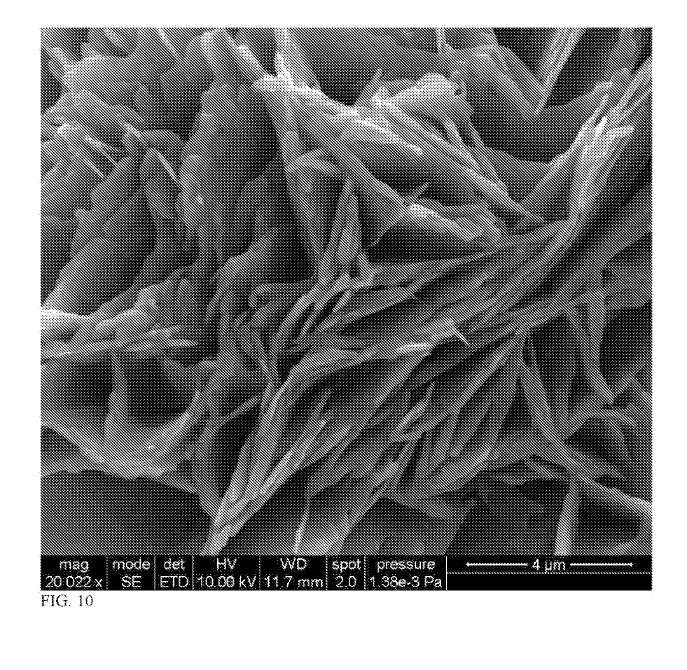

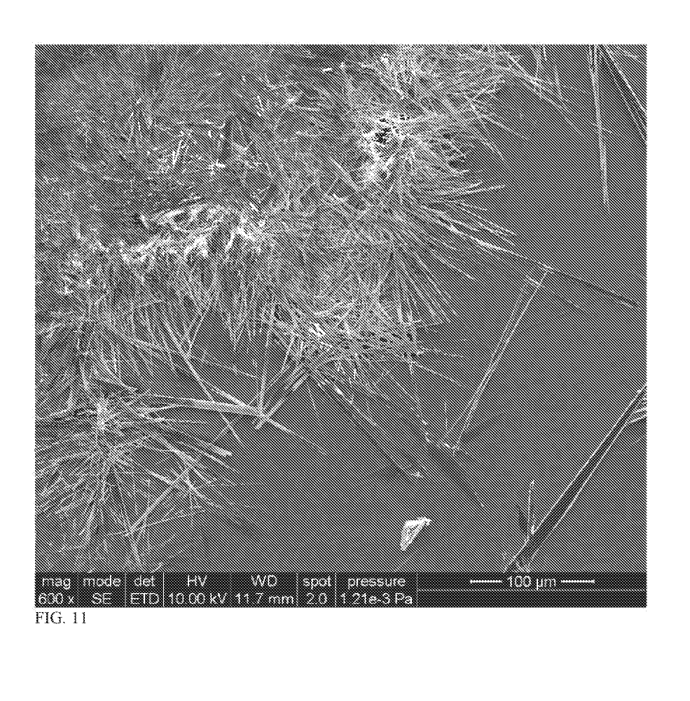

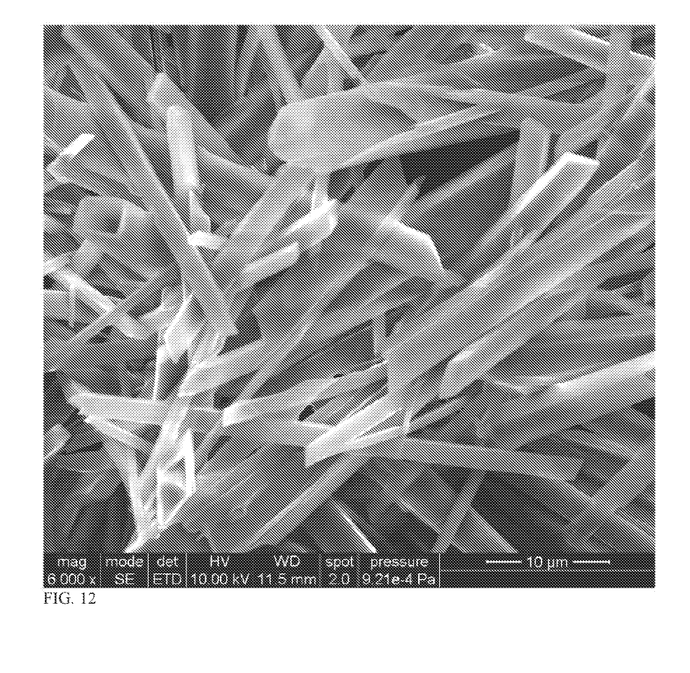

FIG. 10 also depicts an SEM image of a surface of a porous film. This image can illustrate how the terminology described herein may be applied to the structure of protrusions or particles in a porous film. Although not exhaustive, the following descriptions also apply to at least one of the protrusions or particles in this figure: nanoflakes and pseudoplanar.

A scale bar of 4 μm is indicated in the SEM of FIG. 10, which may provide an indication of the size of the nanoparticles, nanoprotrusions, or voids of the film. This figure shows that a substantial number of particles or voids may have an x, y, and/or z dimension in the range of about 1 μm to about 20 μm.

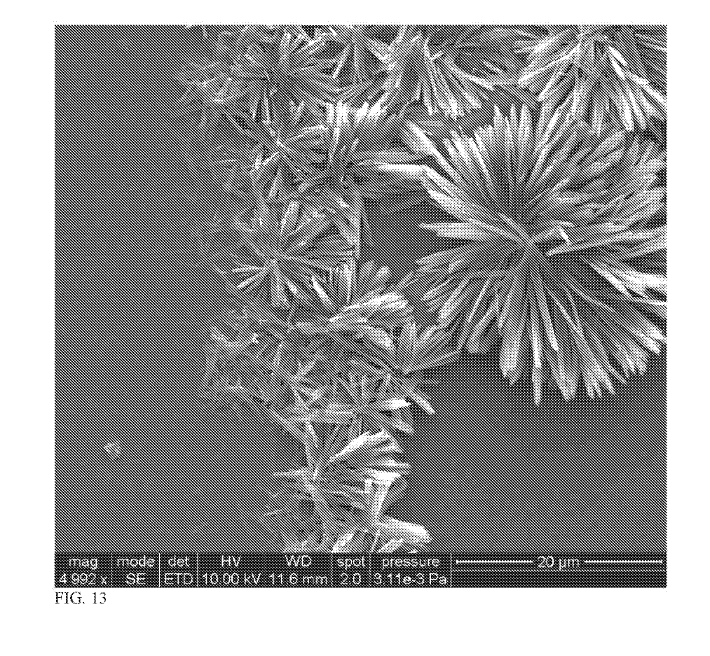

FIG. 11 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane, the xz plane, and/or the yz plane: substantially linear. Although not exhaustive, the following other descriptions may also apply to at least one of the protrusions or particles in this figure: fiber-shaped and needlelike.

A scale bar of 100 μm is indicated in the SEM of FIG. 11, which may provide an indication of the size of the nanoparticles, nanoprotrusions, or voids of the film. This figure shows that a substantial number of particles or voids may have an x, y, and/or z dimension in the range of about 20 μm to about 1000 μm.

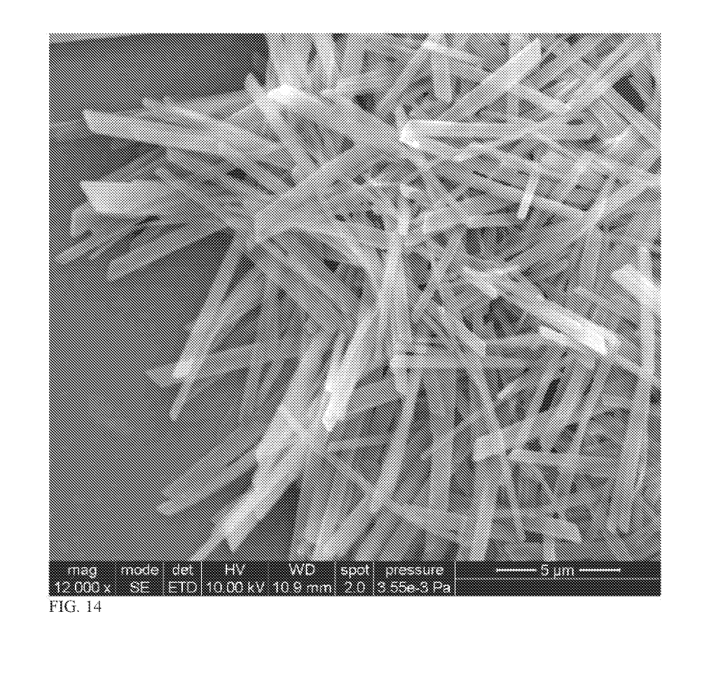

FIG. 12 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear, pseudo-parallelogramatic, and substantially parallelogramatic. Although not exhaustive, the following descriptions may apply to at least one of the protrusions or particles in this figure when viewed in the yz plane: substantially rectangular, substantially linear, pseudo-parallelogramatic, and substantially parallelogramatic. Although not exhaustive, the following other descriptions may also apply to at least one of the protrusions or particles in this figure: fiber-shaped and needlelike.

A scale bar of 10 μm is indicated in the SEM of FIG. 12, which may provide an indication of the size of the nanoparticles, nanoprotrusions, or voids of the film. This figure shows that a substantial number of particles or voids may have an x, y, and/or z dimension in the range of about 1 μm to about 100 μm.

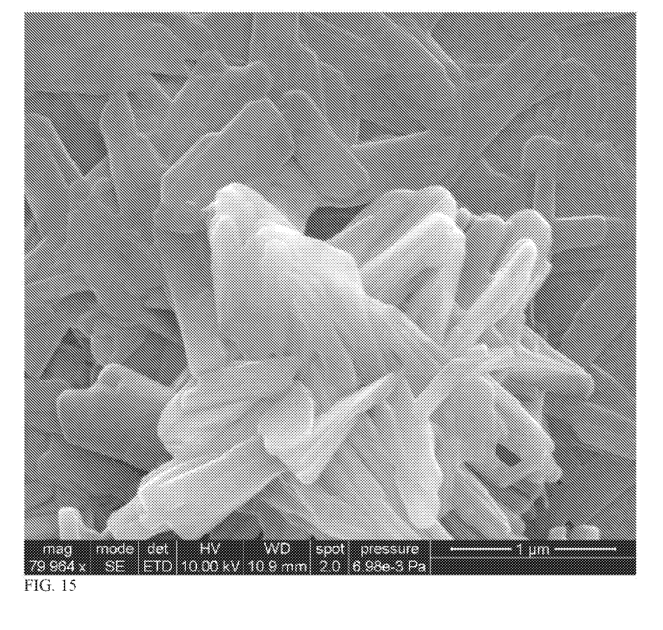

FIG. 13 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear. Although not exhaustive, the following descriptions may apply to at least one of the protrusions or particles in this figure when viewed in the yz plane: substantially linear. Although not exhaustive, the following other descriptions may also apply to at least one of the protrusions or particles in this figure: fiber-shaped, needlelike, and pseudoplanar.

A scale bar of 20 μm is indicated in the SEM of FIG. 13, which may provide an indication of the size of the nanoparticles, nanoprotrusions, or voids of the film. This figure shows that a substantial number of particles or voids may have an x, y, and/or z dimension in the range of about 2 μm to about 100 μm.

FIG. 13 also shows that the particles or protrusions form aggregates having a pseudofloral arrangement. For example, the manner in which some of the particles protrude from a common central area provides an appearance that is recognizable as similar to a flower. For example, the manner in which some of the particles generally radiate from a common central area also provides an appearance that is recognizable as similar to a flower. A substantial number of these pseudofloral aggregates may have a diameter in the range of about 10 μm to about 50 μm.

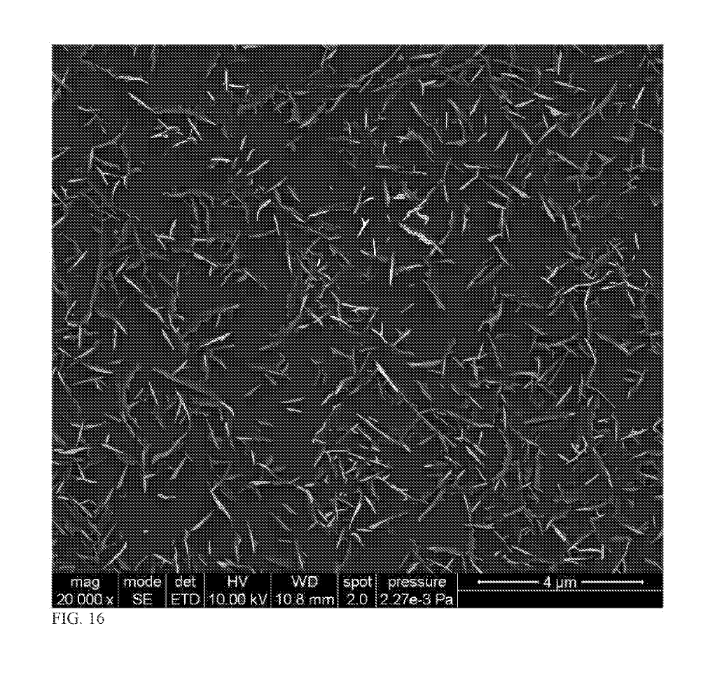

FIG. 14 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear, substantially parallelogramatic, and pseudo-parallelogramatic. Although not exhaustive, the following descriptions may apply to at least one of the protrusions or particles in this figure when viewed in the yz plane: substantially linear, substantially parallelogramatic, and pseudo-parallelogramatic. Although not exhaustive, the following other descriptions may also apply to at least one of the protrusions or particles in this figure: fiber-shaped, needlelike, and pseudoplanar.

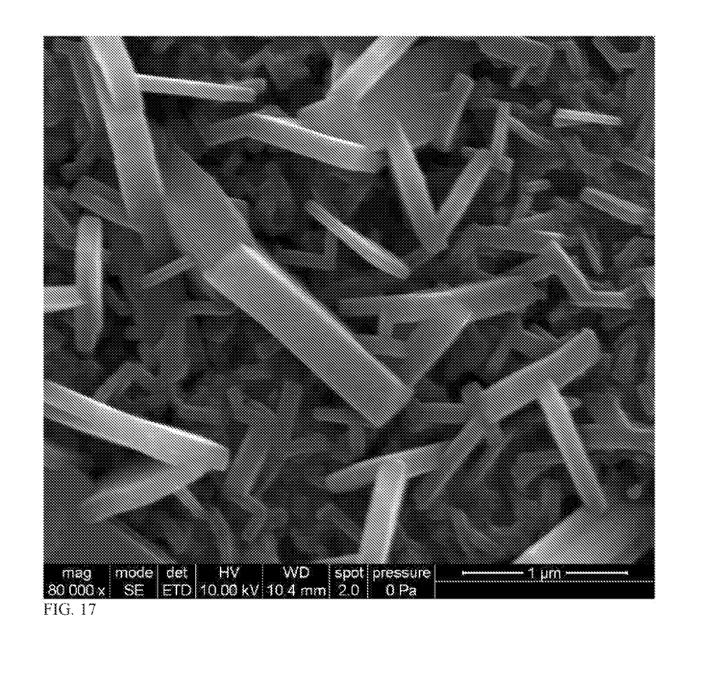

A scale bar of 5 μm is indicated in the SEM of FIG. 15, which may provide an indication of the size of the nanoparticles, nanoprotrusions, or voids of the film. This figure shows that a substantial number of particles or voids may have an x, y, and/or z dimension in the range of about 0.5 μm to about 50 μm.

FIG. 15 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: pseudoplanar.

A scale bar of 1 μm is indicated in the SEM of FIG. 15, which may provide an indication of the size of the nanoparticles, nanoprotrusions, or voids of the film. This figure shows that a substantial number of particles or voids may have an x, y, and/or z dimension in the range of about 0.1 μm to about 5 μm.

FIG. 16 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear. Although not exhaustive, the following descriptions may apply to at least one of the protrusions or particles in this figure when viewed in the yz plane: substantially linear. Although not exhaustive, the following other descriptions may also apply to at least one of the protrusions or particles in this figure: needlelike and pseudoplanar.

A scale bar of 4 μm is indicated in the SEM of FIG. 16, which may provide an indication of the size of the nanoparticles, nanoprotrusions, or voids of the film. This figure shows that a substantial number of particles or voids may have an x, y, and/or z dimension in the range of about 0.1 μm to about 10 μm.

FIG. 17 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially linear, pseudo-parallelogramatic, substantially parallelogramatic, at least one substantially right angle, and substantially all substantially right angles. Although not exhaustive, the following descriptions may apply to at least one of the protrusions or particles in this figure when viewed in the yz plane: substantially rectangular, substantially linear, pseudo-parallelogramatic, substantially parallelogramatic, at least one substantially right angle, and substantially all substantially right angles. Although not exhaustive, the following other descriptions may also apply to at least one of the protrusions or particles in this figure: nanoflake, fiber-shaped, and pseudoplanar.

A scale bar of 1 μm is indicated in the SEM of FIG. 17, which may provide an indication of the size of the nanoparticles, nanoprotrusions, or voids of the film. This figure shows that a substantial number of particles or voids may have an x, y, and/or z dimension in the range of about 0.1 μm to about 5 μm.

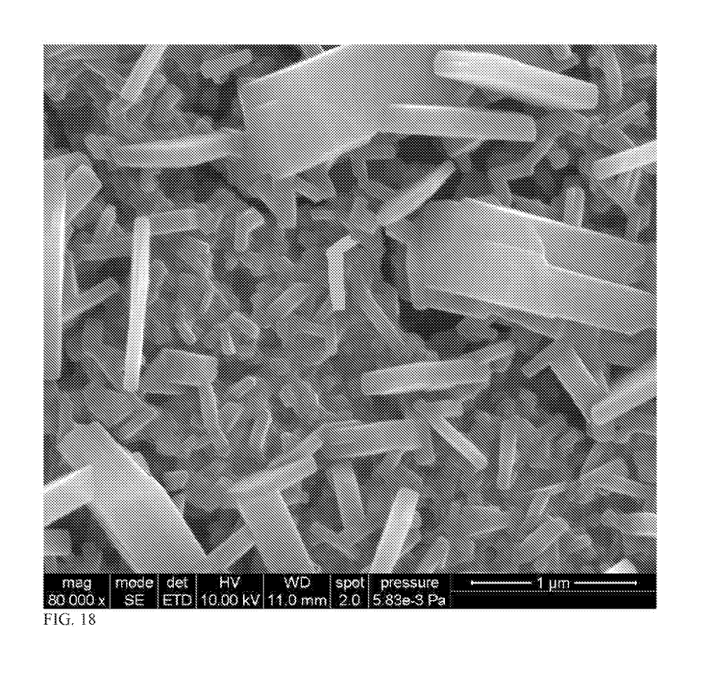

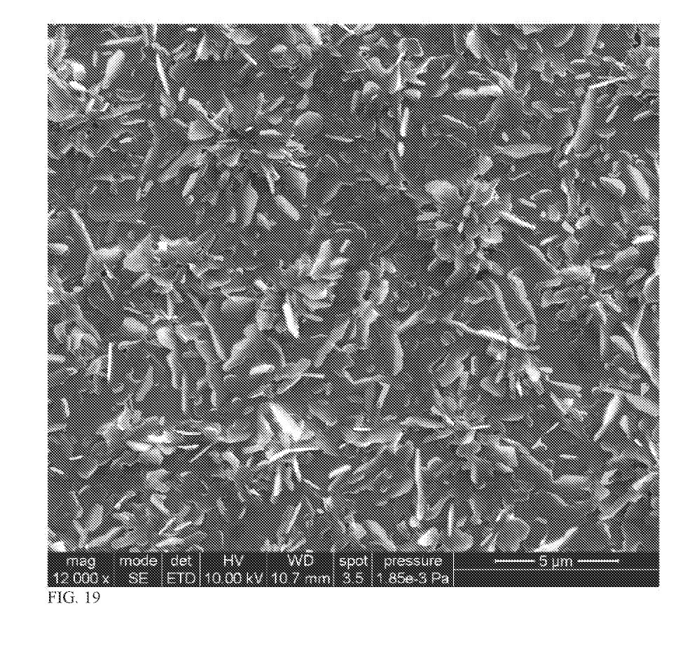

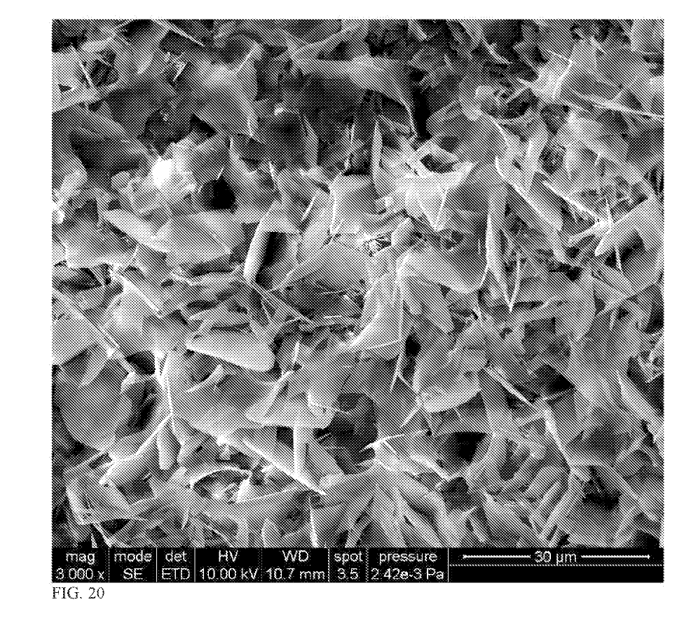

FIG. 18 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, pseudo-parallelogramatic, substantially parallelogramatic, and substantially all substantially right angles. Although not exhaustive, the following descriptions may apply to at least one of the protrusions or particles in this figure when viewed in the yz plane: substantially linear, substantially rectangular, substantially parallelogramatic, and pseudo-parallelogramatic. Although not exhaustive, the following other descriptions may also apply to at least one of the protrusions or particles in this figure: pseudoplanar.

A scale bar of 1 μm is indicated in the SEM of FIG. 18, which may provide an indication of the size of the nanoparticles, nanoprotrusions, or voids of the film. This figure shows that a substantial number of particles or voids may have an x, y, and/or z dimension in the range of about 0.1 μm to about 20 μm.

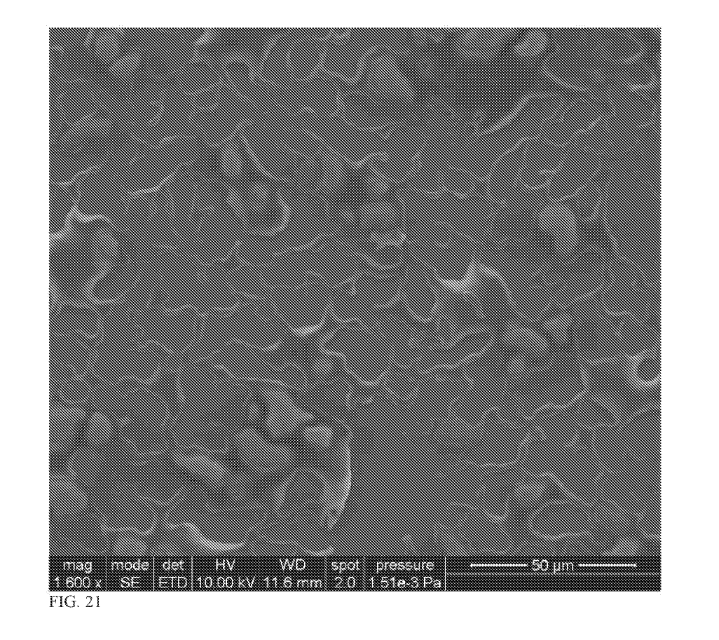

FIG. 19 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: nanoflake and pseudoplanar.

A scale bar of 5 μm is indicated in the SEM of FIG. 19, which may provide an indication of the size of the nanoparticles, nanoprotrusions, or voids of the film. This figure shows that a substantial number of particles or voids may have an x, y, and/or z dimension in the range of about 1 μm to about 20 μm.

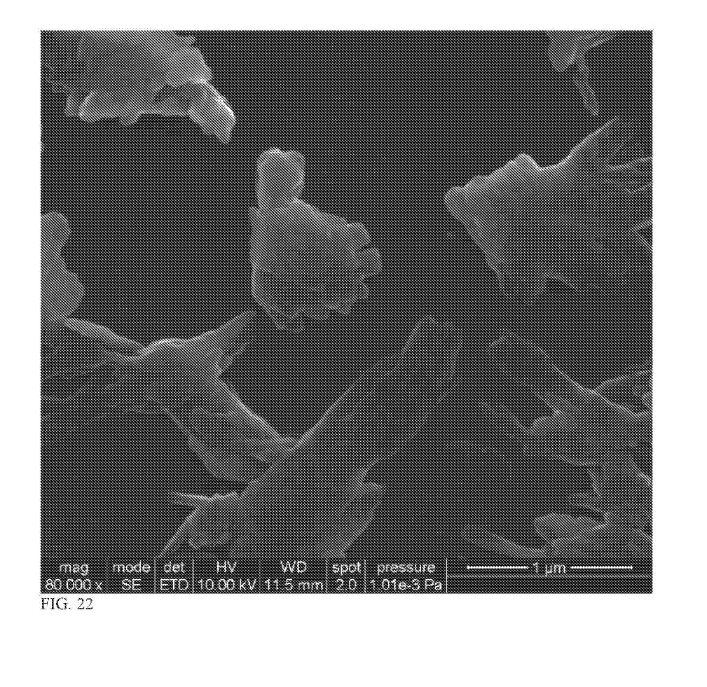

FIG. 20 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the yz plane: substantially linear. Although not exhaustive, the following other descriptions may also apply to at least one of the protrusions or particles in this figure: nanoflake and pseudoplanar.

A scale bar of 30 μm is indicated in the SEM of FIG. 20, which may provide an indication of the size of the nanoparticles, nanoprotrusions, or voids of the film. This figure shows that a substantial number of particles or voids may have an x, y, and/or z dimension in the range of about 1 μm to about 50 μm.

FIG. 21 also depicts an SEM image of a surface of a porous film. This image can illustrate how the terminology described herein may be applied to the structure of protrusions or particles in a porous film. Although not exhaustive, the following description may also apply to at least one of the protrusions or particles in this figure: pseudoplanar.

A scale bar of 50 μm is indicated in the SEM of FIG. 21, which may provide an indication of the size of the nanoparticles, nanoprotrusions, or voids of the film. This figure shows that a substantial number of particles or voids may have an x, y, and/or z dimension in the range of about 1 μm to about 200 μm.

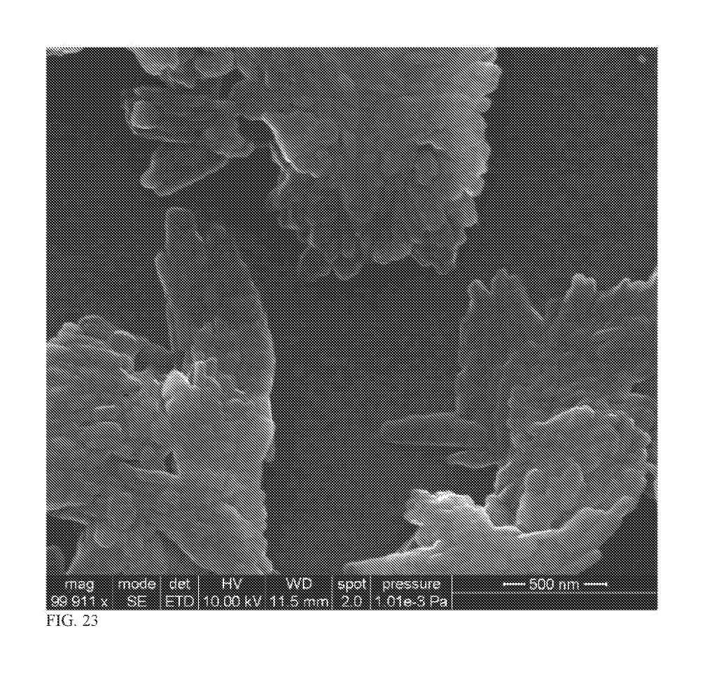

FIG. 22 also depicts an SEM image of a surface of a porous film. This image can illustrate how the terminology described herein may be applied to the structure of protrusions or particles in a porous film. Although not exhaustive, the following description may apply to at least one of the protrusions or particles in this figure: pseudoplanar.

A scale bar of 1 μm is indicated in the SEM of FIG. 22, which may provide an indication of the size of the nanoparticles, nanoprotrusions, or voids of the film. This figure shows that a substantial number of particles or voids may have an x, y, and/or z dimension in the range of about 0.1 μm to about 5 μm.

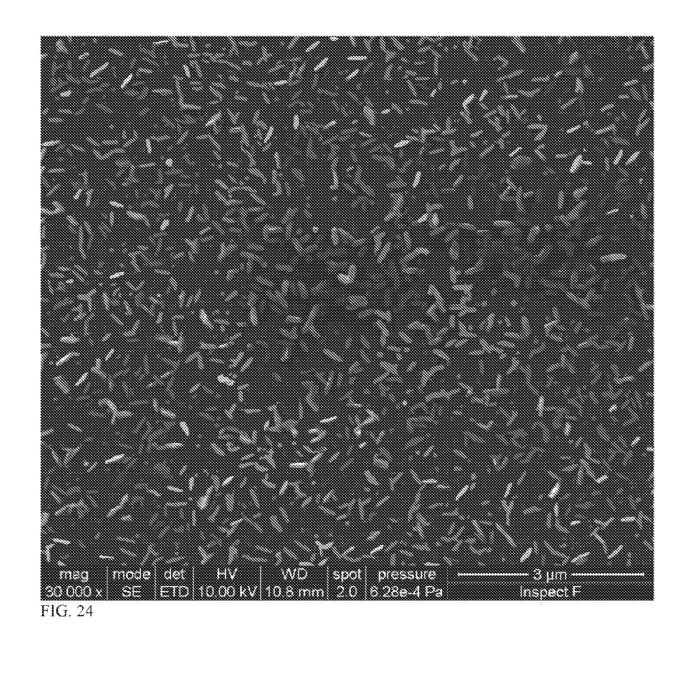

FIG. 23 also depicts an SEM image of a surface of a porous film. This image can illustrate how the terminology described herein may be applied to the structure of protrusions or particles in a porous film. Although not exhaustive, the following description may apply to at least one of the protrusions or particles in this figure: pseudoplanar.

A scale bar of 500 nm is indicated in the SEM of FIG. 23, which may provide an indication of the size of the nanoparticles, nanoprotrusions, or voids of the film. This figure shows that a substantial number of particles or voids may have an x, y, and/or z dimension in the range of about 50 nm to about 5 μm.

FIG. 24 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy, xy, and/the yz plane: substantially oval, substantially elliptical, and substantially circular. Although not exhaustive, the following other descriptions may also apply to at least one of the protrusions or particles in this figure: rod-shaped, substantially capsule-shaped.

A scale bar of 3 μm is indicated in the SEM of FIG. 24, which may provide an indication of the size of the nanoparticles, nanoprotrusions, or voids of the film. This figure shows that a substantial number of particles or voids may have an x, y, and/or z dimension in the range of about 0.01 μm to about 1 μm.

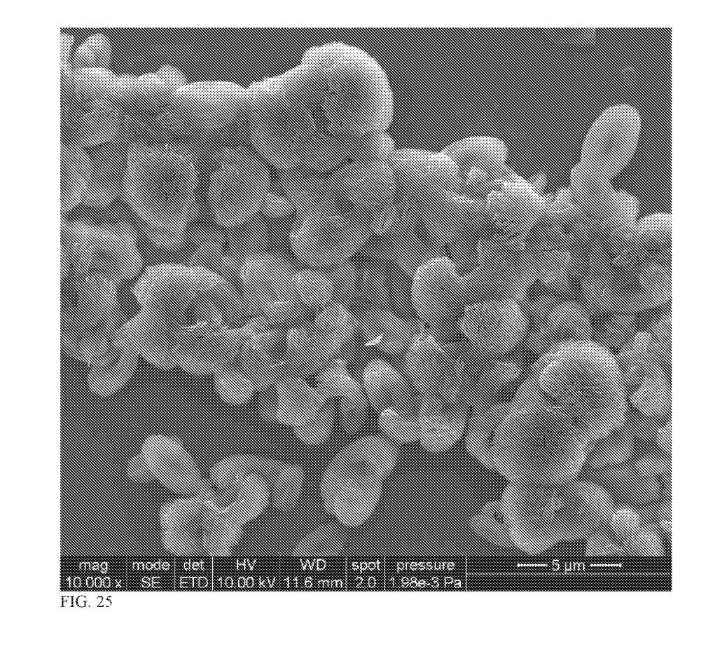

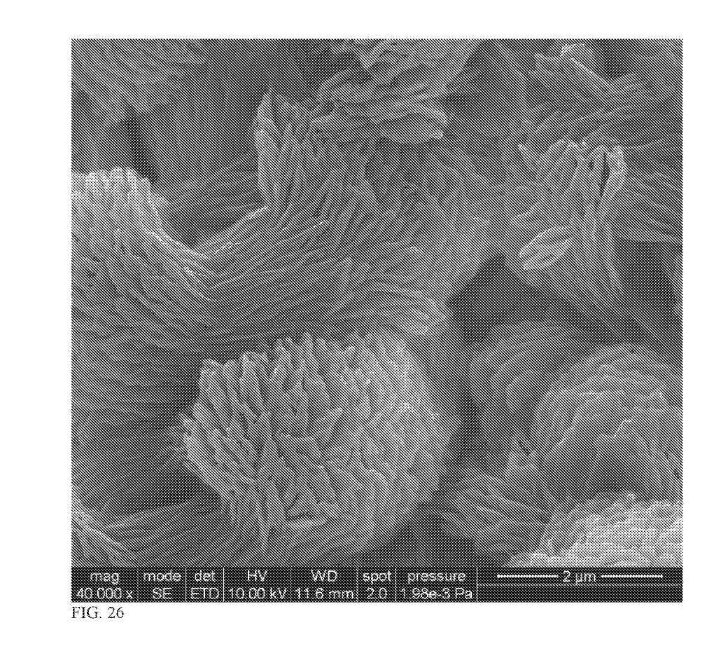

FIG. 25 also depicts an SEM image of a surface of a porous film. This image can illustrate how the terminology described herein may be applied to the structure of protrusions or particles in a porous film. Although not exhaustive, the following descriptions may apply to at least one of the protrusions, particles, and/or aggregates thereof: fiber-shaped.

A scale bar of 5 μm is indicated in the SEM of FIG. 25, which may provide an indication of the size of the nanoparticles, nanoprotrusions, or voids of the film. This figure shows that a substantial number of particles or voids may have an x, y, and/or z dimension in the range of about 0.01 μm to about 20 μm.

FIG. 25 also comprises aggregates of nanoparticles or nanoprotrusion having a fiber bundle configuration. In some embodiments, the aggregates may be described as having a center-bound fiber bundle configuration in that they may resemble a bundle of fibers having a strap or binding in the center of the bundle holding it together, such that the ends diverge more than the center of the bundle.

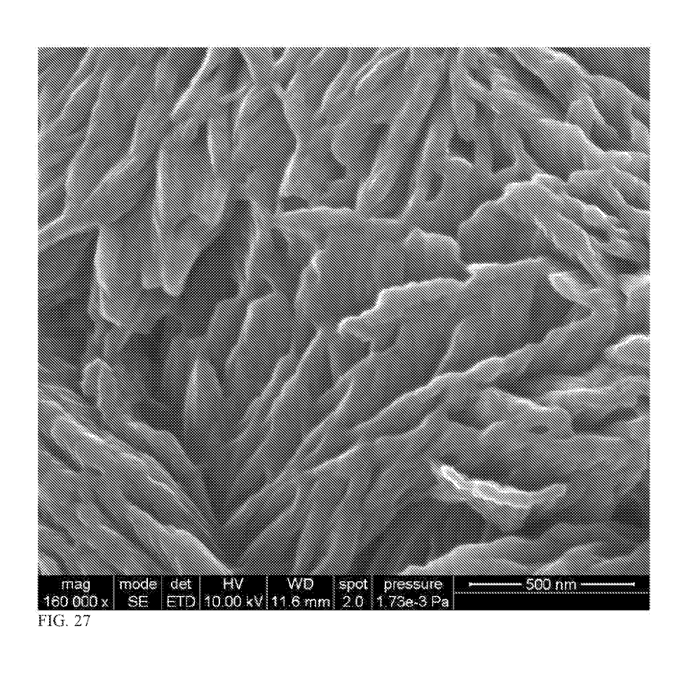

FIG. 26 also depicts an SEM image of a surface of a porous film. This image can illustrate how the terminology described herein may be applied to the structure of protrusions or particles in a porous film. Although not exhaustive, the following descriptions may apply to at least one of the protrusions, particles, and/or aggregates thereof: fiber-shaped.

A scale bar of 2 μm is indicated in the SEM of FIG. 26, which may provide an indication of the size of the nanoparticles, nanoprotrusions, or voids of the film. This figure shows that a substantial number of particles or voids may have an x, y, and/or z dimension in the range of about 0.01 μm to about 10 μm.

FIG. 26 also comprises aggregates of nanoparticles or nanoprotrusion having a fiber bundle configuration and/or a center-bound fiber bundle configuration.

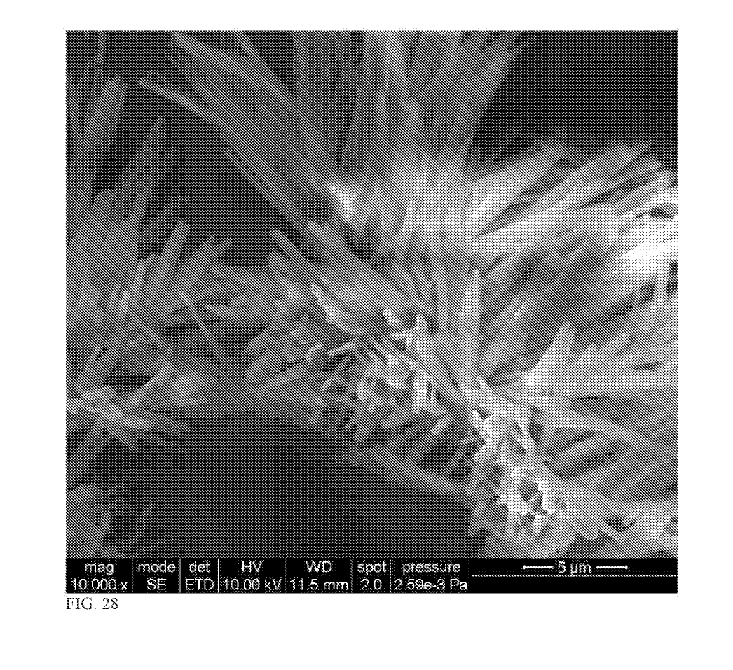

FIG. 27 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: fiber-shaped and pseudoplanar.

A scale bar of 500 nm is indicated in the SEM of FIG. 27, which may provide an indication of the size of the nanoparticles, nanoprotrusions, or voids of the film. This figure shows that a substantial number of particles or voids may have an x, y, and/or z dimension in the range of about 5 nm to about 5 μm.

FIG. 28 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear. Although not exhaustive, the following other descriptions may also apply to at least one of the protrusions or particles in this figure: needlelike and fiber-shaped.

A scale bar of 5 μm is indicated in the SEM of FIG. 28, which may provide an indication of the size of the nanoparticles, nanoprotrusions, or voids of the film. This figure shows that a substantial number of particles or voids may have an x, y, and/or z dimension in the range of about 0.1 μm to about 100 μm.

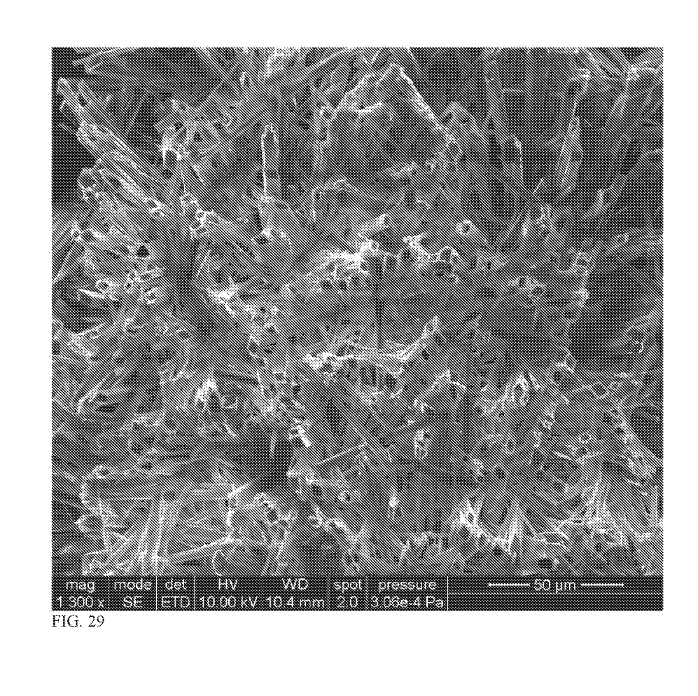

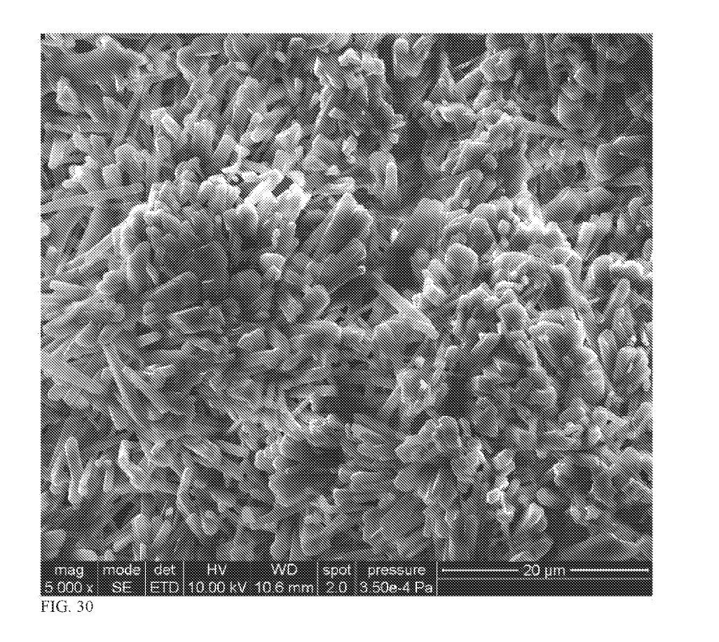

FIG. 29 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially parallelogramatic, at least one substantially right angle, and substantially linear. Although not exhaustive, the following descriptions may apply to at least one of the protrusions or particles in this figure when viewed in the yz plane: substantially parallelogramatic. Although not exhaustive, the following other descriptions may also apply to at least one of the protrusions or particles in this figure: needlelike and fiber-shaped.

A scale bar of 50 μm is indicated in the SEM of FIG. 29, which may provide an indication of the size of the nanoparticles, nanoprotrusions, or voids of the film. This figure shows that a substantial number of particles or voids may have an x, y, and/or z dimension in the range of about 1 μm to about 500 μm.

FIG. 30 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: pseudo-parallelogramatic, substantially parallelogramatic, and substantially linear. Although not exhaustive, the following descriptions may apply to at least one of the protrusions or particles in this figure when viewed in the yz plane: substantially parallelogramatic. Although not exhaustive, the following other descriptions may also apply to at least one of the protrusions or particles in this figure: needlelike, and fiber-shaped.

A scale bar of 20 μm is indicated in the SEM of FIG. 30, which may provide an indication of the size of the nanoparticles, nanoprotrusions, or voids of the film. This figure shows that a substantial number of particles or voids may have an x, y, and/or z dimension in the range of about 0.1 μm to about 150 μm.

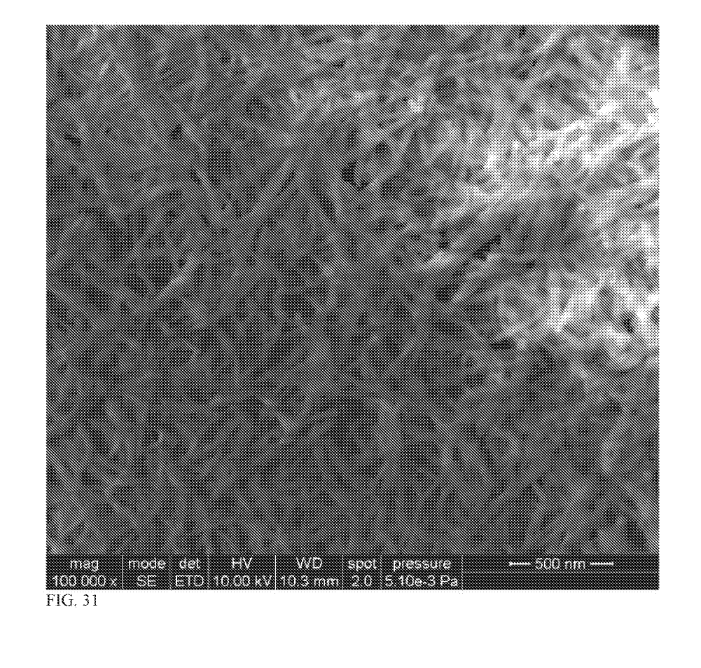

FIG. 31 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: fiber-shaped.

A scale bar of 500 nm is indicated in the SEM of FIG. 31, which may provide an indication of the size of the nanoparticles, nanoprotrusions, or voids of the film. This figure shows that a substantial number of particles or voids may have an x, y, and/or z dimension in the range of about 10 nm to about 5 μm.

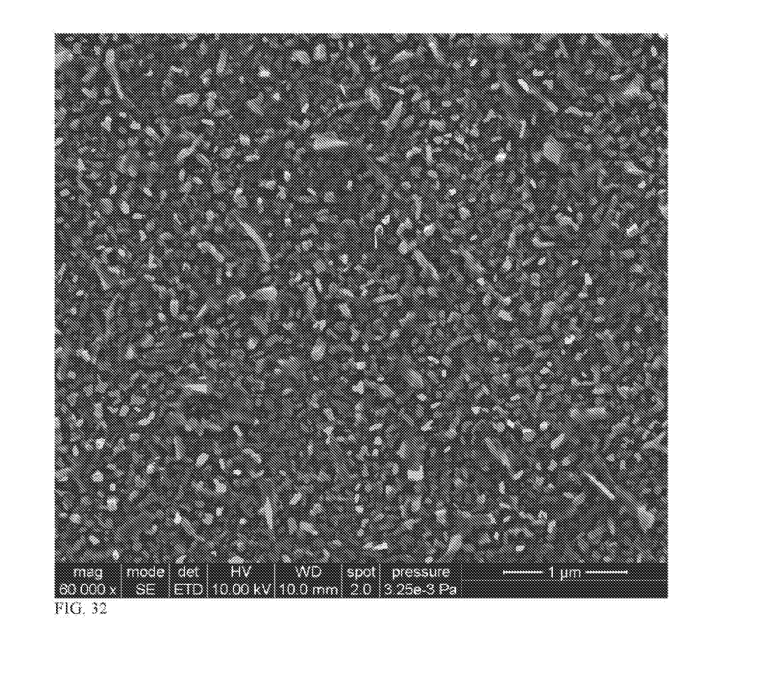

FIG. 32 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy, xz, or the yz plane: substantially rectangular, at least one substantially right angle, substantially all substantially right angles, and substantially linear. Although not exhaustive, the following descriptions may apply to at least one of the protrusions or particles in this figure when viewed in the yz plane: substantially linear, substantially parallelogramatic, and pseudo-parallelogramatic. Although not exhaustive, the following other descriptions may also apply to at least one of the protrusions or particles in this figure: granular.

A scale bar of 1 μm is indicated in the SEM of FIG. 32, which may provide an indication of the size of the nanoparticles, nanoprotrusions, or voids of the film. This figure shows that a substantial number of particles or voids may have an x, y, and/or z dimension in the range of about 0.01 μm to about 5 μm.

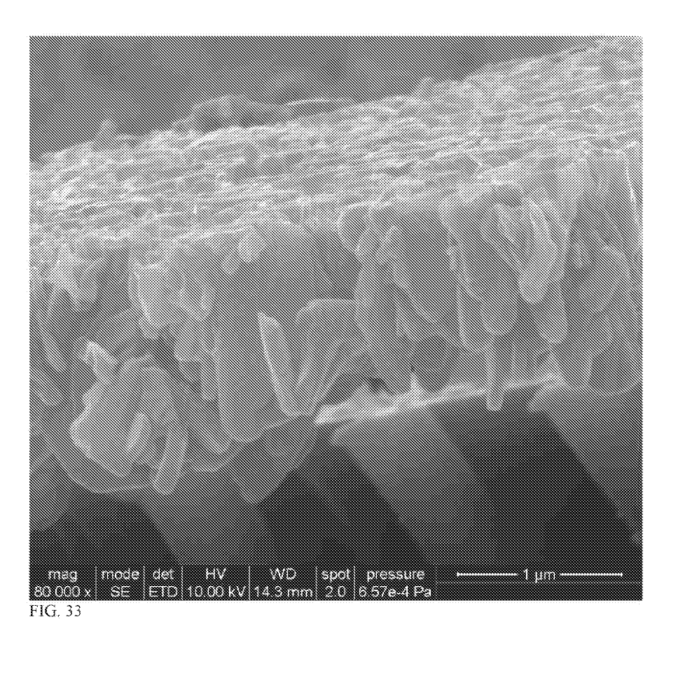

FIG. 33 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: nanoflake and pseudoplanar.

A scale bar of 1 μm is indicated in the SEM of FIG. 33, which may provide an indication of the size of the nanoparticles, nanoprotrusions, or voids of the film. This figure shows that a substantial number of particles or voids may have an x, y, and/or z dimension in the range of about 0.1 μm to about 20 μm.

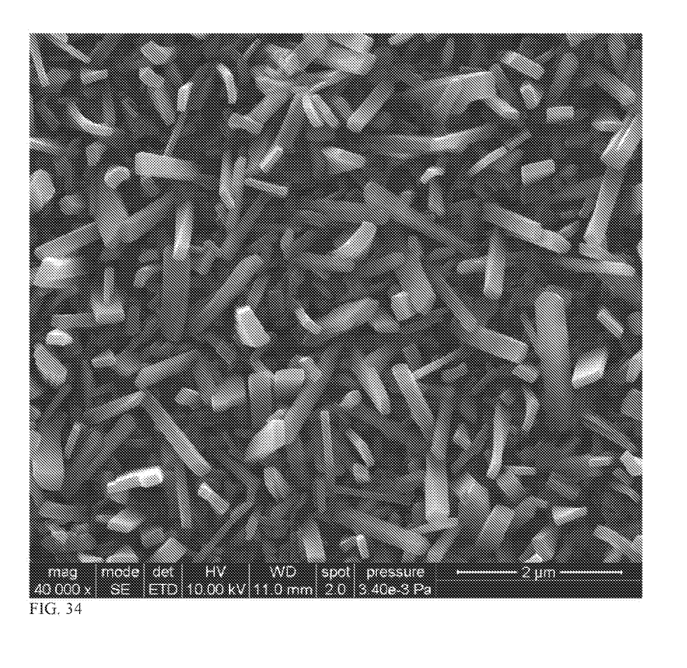

FIG. 34 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially linear, and substantially rectangular. Although not exhaustive, the following other descriptions may also apply to at least one of the protrusions or particles in this figure: fiber-shaped and ribbon-shaped.

A scale bar of 2 μm is indicated in the SEM of FIG. 34, which may provide an indication of the size of the nanoparticles, nanoprotrusions, or voids of the film. This figure shows that a substantial number of particles or voids may have an x, y, and/or z dimension in the range of about 0.1 μm to about 10 μm.

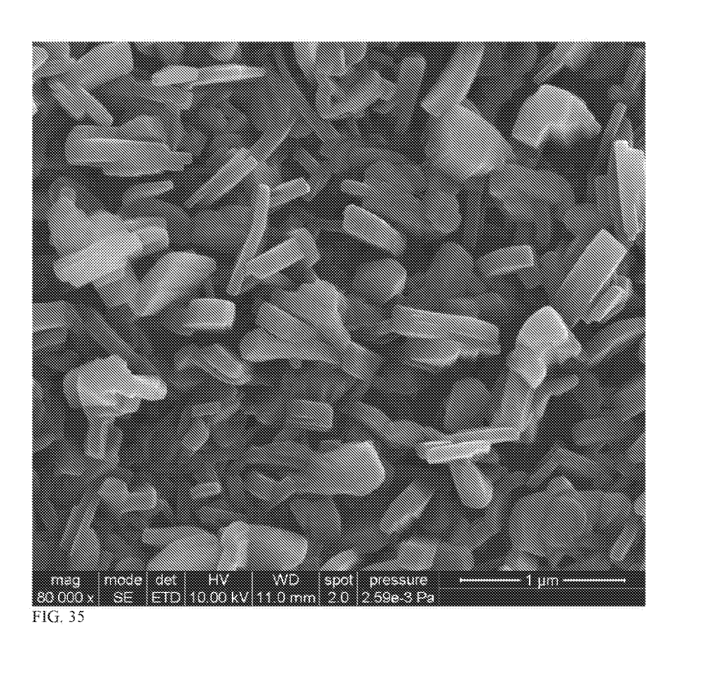

FIG. 35 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially linear, substantially rectangular, at least one substantially right angle, and substantially all substantially right angle. Although not exhaustive, the following other descriptions may also apply to at least one of the protrusions or particles in this figure: fiber-shaped and granular.

A scale bar of 1 μm is indicated in the SEM of FIG. 35, which may provide an indication of the size of the nanoparticles, nanoprotrusions, or voids of the film. This figure shows that a substantial number of particles or voids may have an x, y, and/or z dimension in the range of about 0.01 μm to about 10 μm.

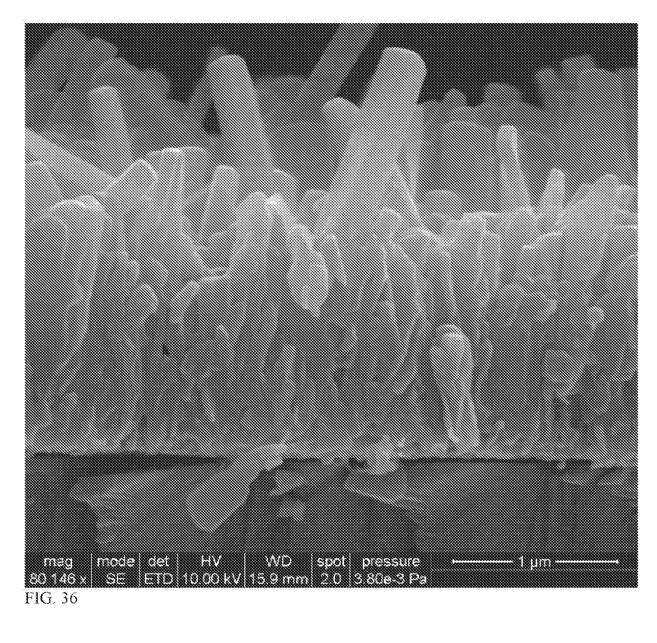

FIG. 36 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially rectangular. Although not exhaustive, the following other descriptions may also apply to at least one of the protrusions or particles in this figure: fiber-shaped and pseudoplanar.

A scale bar of 1 μm is indicated in the SEM of FIG. 36, which may provide an indication of the size of the nanoparticles, nanoprotrusions, or voids of the film. This figure shows that a substantial number of particles or voids may have an x, y, and/or z dimension in the range of about 0.01 μm to about 10 μm.

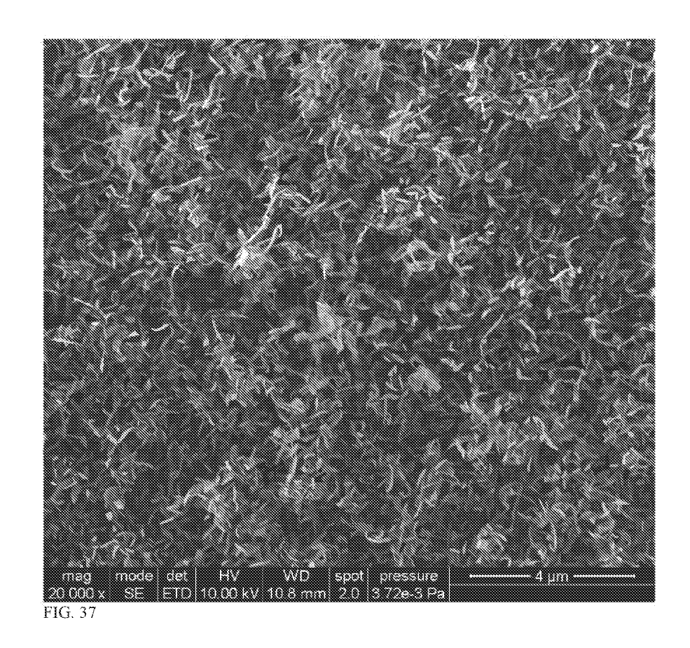

FIG. 37 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear. Although not exhaustive, the following other descriptions may also apply to at least one of the protrusions or particles in this figure: rod-shaped and fiber-shaped.

A scale bar of 4 μm is indicated in the SEM of FIG. 37, which may provide an indication of the size of the nanoparticles, nanoprotrusions, or voids of the film. This figure shows that a substantial number of particles or voids may have an x, y, and/or z dimension in the range of about 0.05 μm to about 10 μm.

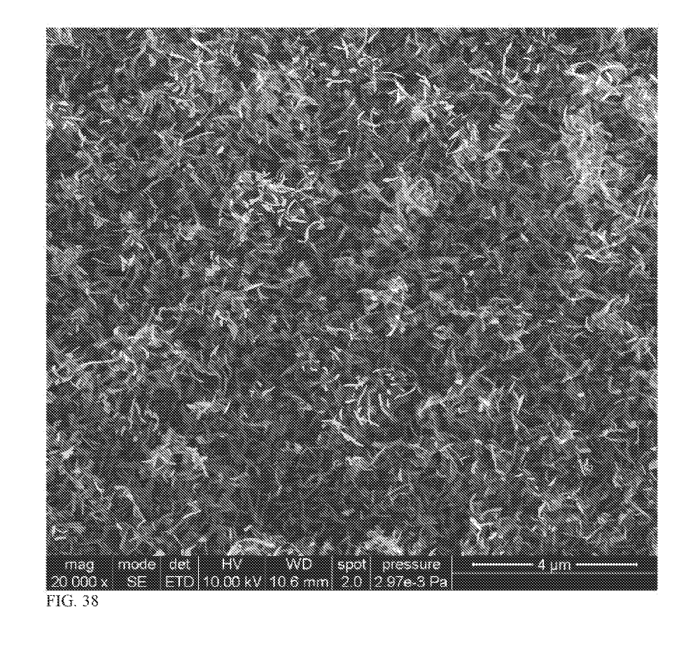

FIG. 38 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially linear. Although not exhaustive, the following other descriptions may also apply to at least one of the protrusions or particles in this figure: rod-shaped and fiber-shaped.

A scale bar of 4 μm is indicated in the SEM of FIG. 38, which may provide an indication of the size of the nanoparticles, nanoprotrusions, or voids of the film. This figure shows that a substantial number of particles or voids may have an x, y, and/or z dimension in the range of about 0.05 μm to about 10 μm.

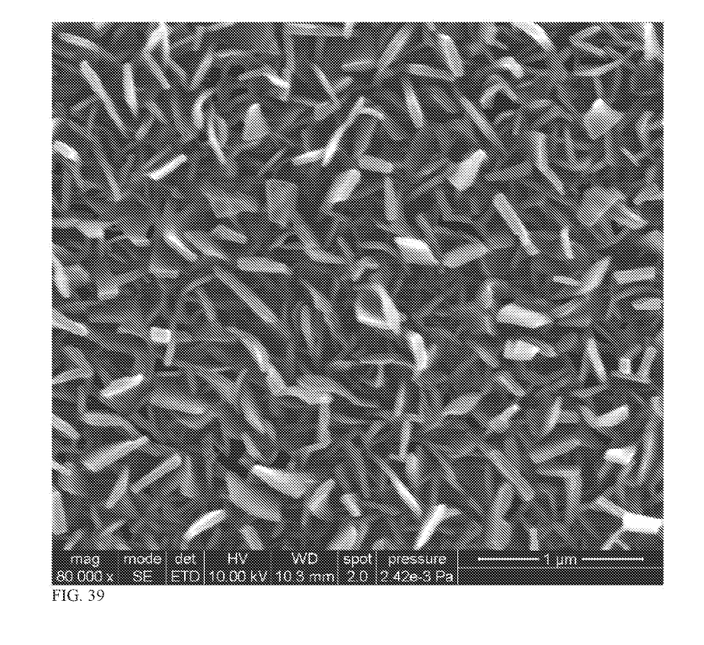

FIG. 39 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially linear, substantially rectangular, at least one substantially right angle, and substantially all substantially right angles Although not exhaustive, the following other descriptions may also apply to at least one of the protrusions or particles in this figure: ribbon-shaped, nanoflake and pseudoplanar.

A scale bar of 1 μm is indicated in the SEM of FIG. 39, which may provide an indication of the size of the nanoparticles, nanoprotrusions, or voids of the film. This figure shows that a substantial number of particles or voids may have an x, y, and/or z dimension in the range of about 0.1 μm to about 20 μm.

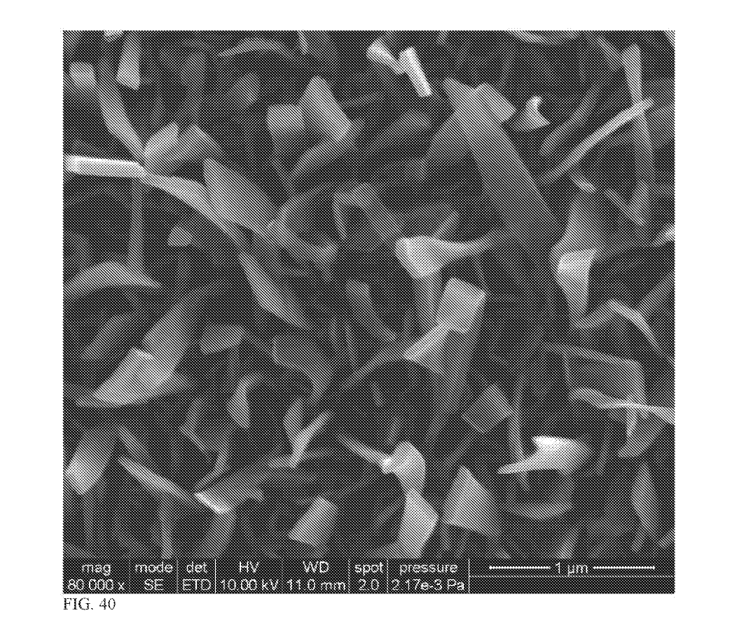

FIG. 40 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, pseudo-parallelogramatic, substantially parallelogramatic, at least one substantially right angle, and substantially all substantially right angles. Although not exhaustive, the following descriptions may apply to at least one of the protrusions or particles in this figure when viewed in the yz plane: substantially linear, substantially parallelogramatic, and pseudo-parallelogramatic. Although not exhaustive, the following other descriptions may also apply to at least one of the protrusions or particles in this figure: ribbon-shaped, fiber-shaped, and pseudoplanar.

A scale bar of 1 μm is indicated in the SEM of FIG. 40, which may provide an indication of the size of the nanoparticles, nanoprotrusions, or voids of the film. This figure shows that a substantial number of particles or voids may have an x, y, and/or z dimension in the range of about 0.01 μm to about 10 μm.

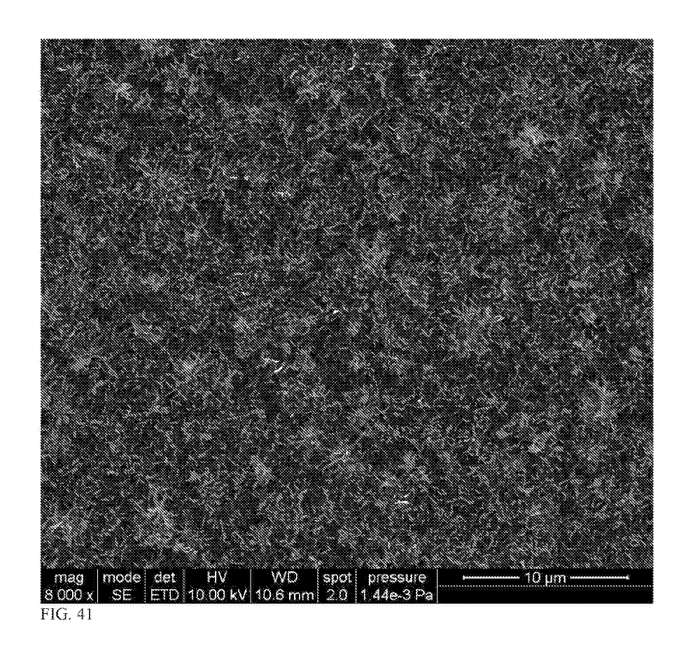

FIG. 41 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: rod-shaped and fiber-shaped.

A scale bar of 10 μm is indicated in the SEM of FIG. 41, which may provide an indication of the size of the nanoparticles, nanoprotrusions, or voids of the film. This figure shows that a substantial number of particles or voids may have an x, y, and/or z dimension in the range of about 0.1 μm to about 10 μm.

FIG. 42 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially linear, at least one substantially right angle, and substantially all substantially right angles. Although not exhaustive, the following other descriptions may also apply to at least one of the protrusions or particles in this figure: fiber-shaped and ribbon shaped.

A scale bar of 1 μm is indicated in the SEM of FIG. 42, which may provide an indication of the size of the nanoparticles, nanoprotrusions, or voids of the film. This figure shows that a substantial number of particles or voids may have an x, y, and/or z dimension in the range of about 0.1 μm to about 5 μm.

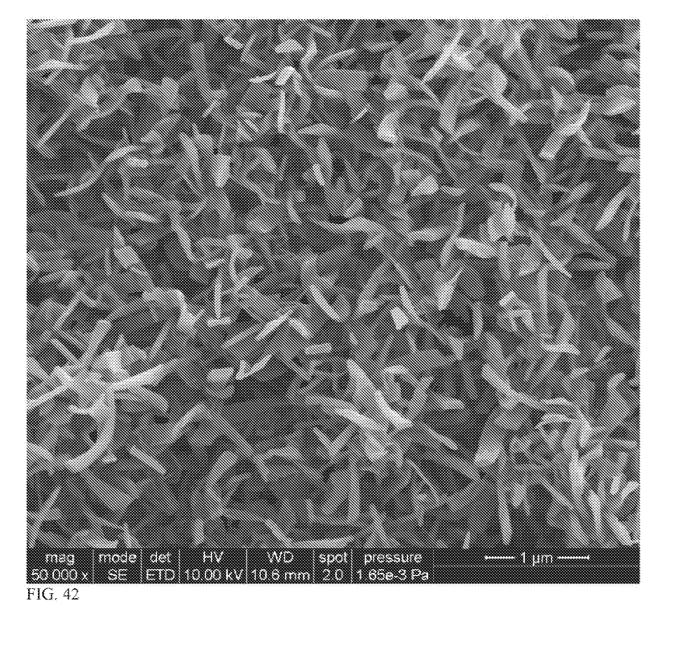

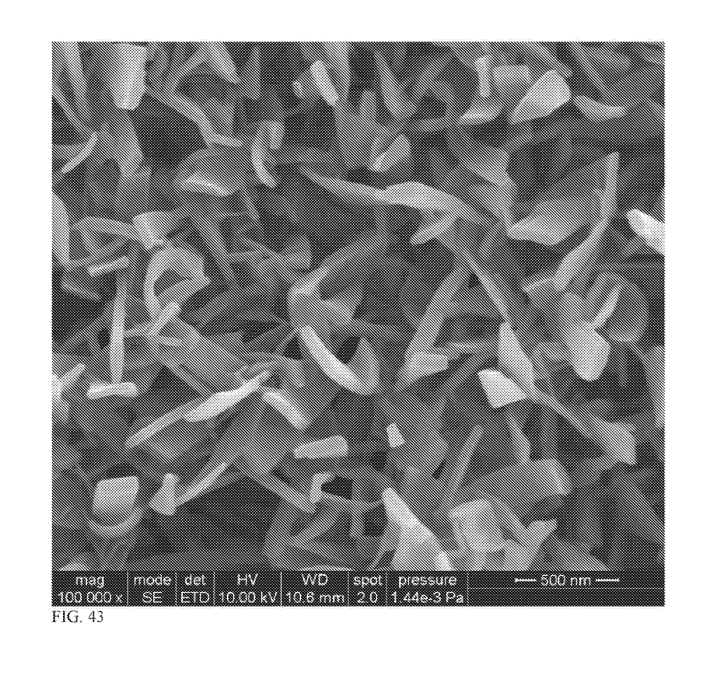

FIG. 43 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially linear, substantially rectangular, at least one substantially right angle, and substantially all substantially right angles. Although not exhaustive, the following other descriptions may also apply to at least one of the protrusions or particles in this figure: nanoflake, ribbon-shaped, and pseudoplanar.

A scale bar of 500 nm is indicated in the SEM of FIG. 43, which may provide an indication of the size of the nanoparticles, nanoprotrusions, or voids of the film. This figure shows that a substantial number of particles or voids may have an x, y, and/or z dimension in the range of about 50 nm to about 2 μm.

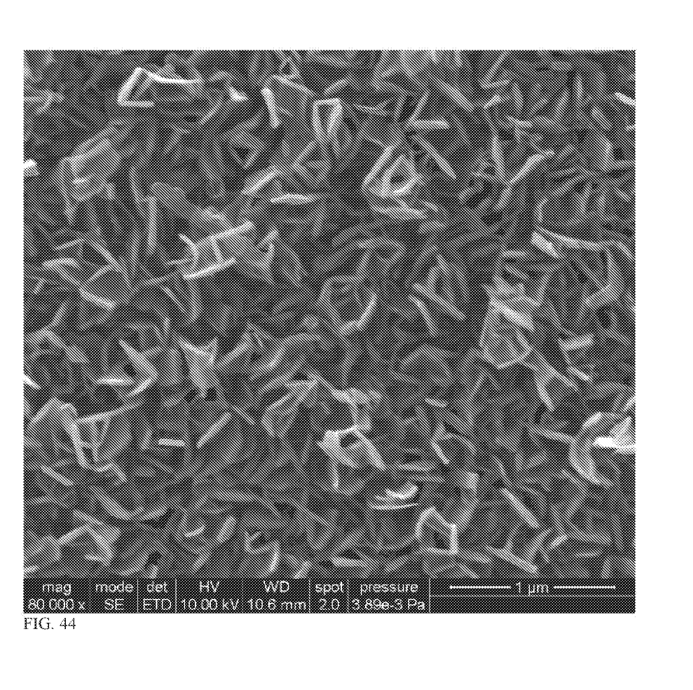

FIG. 44 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially linear, at least one substantially right angle, and substantially all substantially right angles. Although not exhaustive, the following descriptions may apply to at least one of the protrusions or particles in this figure when viewed in the yz plane: substantially rectangular, substantially linear, at least one substantially right angle, and substantially all substantially right angles. Although not exhaustive, the following other descriptions may also apply to at least one of the protrusions or particles in this figure: ribbon-shaped, nanoflake, and pseudoplanar.

A scale bar of 1 μm is indicated in the SEM of FIG. 44, which may provide an indication of the size of the nanoparticles, nanoprotrusions, or voids of the film. This figure shows that a substantial number of particles or voids may have an x, y, and/or z dimension in the range of about 0.01 μm to about 1 μm.

FIG. 45 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially linear, at least one substantially right angle, and substantially all substantially right angles. Although not exhaustive, the following descriptions may apply to at least one of the protrusions or particles in this figure when viewed in the yz plane: substantially rectangular, substantially linear, at least one substantially right angle, and substantially all substantially right angles. Although not exhaustive, the following other descriptions may also apply to at least one of the protrusions or particles in this figure: ribbon-shaped, nanoflake, and pseudoplanar.

A scale bar of 1 μm is indicated in the SEM of FIG. 45, which may provide an indication of the size of the nanoparticles, nanoprotrusions, or voids of the film. This figure shows that a substantial number of particles or voids may have an x, y, and/or z dimension in the range of about 0.1 μm to about 20 μm.

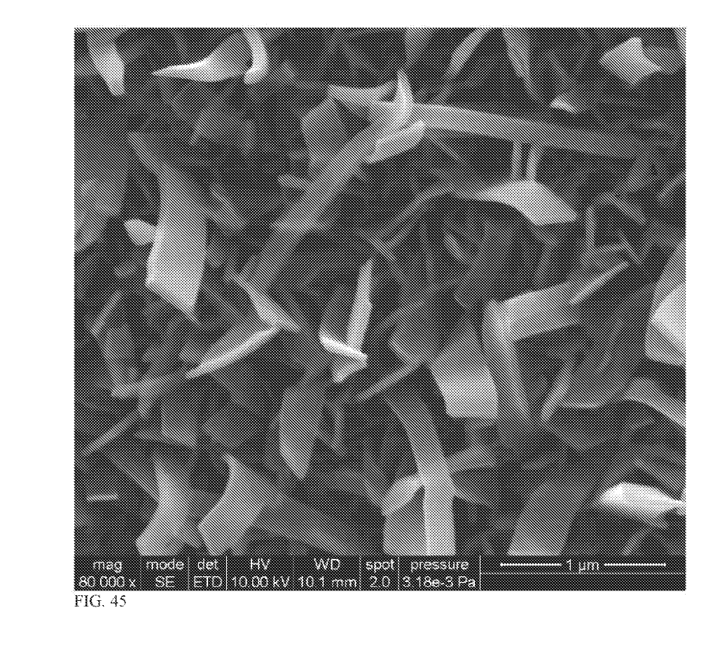

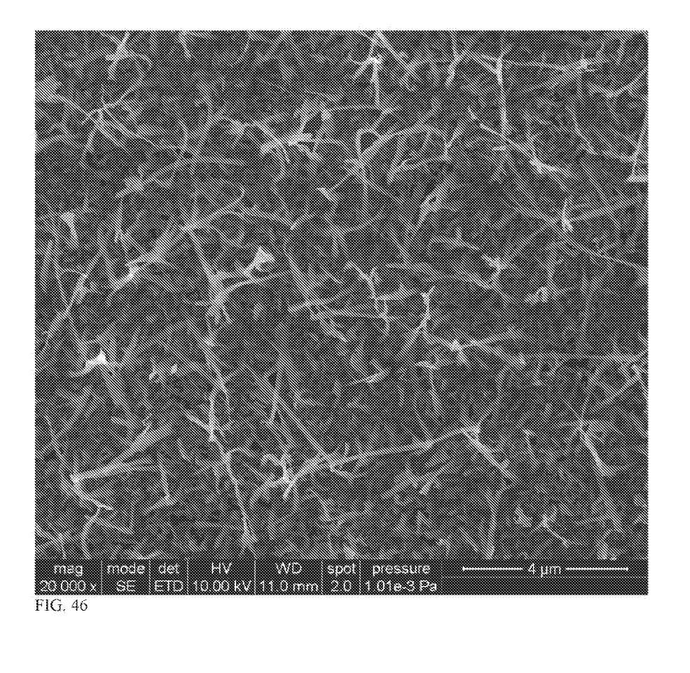

FIG. 46 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, substantially linear, and substantially all substantially right angles. Although not exhaustive, the following other descriptions may also apply to at least one of the protrusions or particles in this figure: ribbon-shaped, fiber-shaped, and pseudoplanar.

A scale bar of 4 μm is indicated in the SEM of FIG. 46, which may provide an indication of the size of the nanoparticles, nanoprotrusions, or voids of the film. This figure shows that a substantial number of particles or voids may have an x, y, and/or z dimension in the range of about 0.05 μm to about 10 μm.

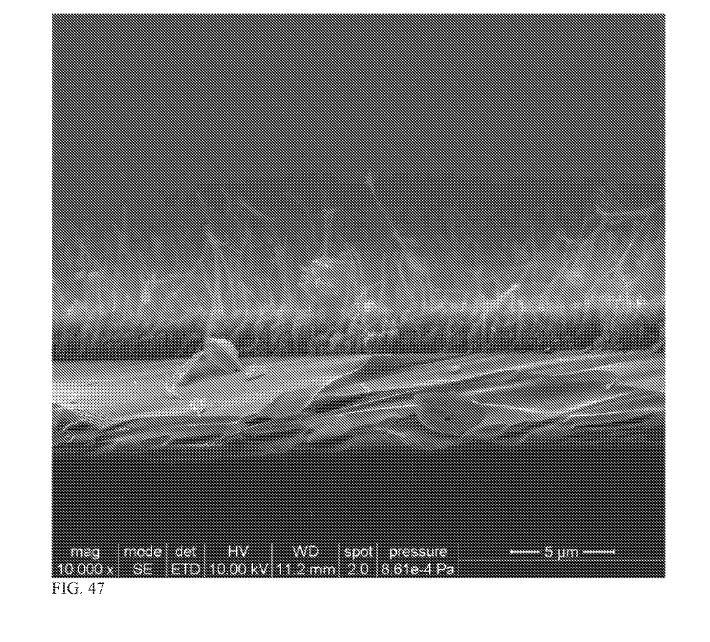

FIG. 47 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: fiber-shaped.

A scale bar of 5 μm is indicated in the SEM of FIG. 47, which may provide an indication of the size of the nanoparticles, nanoprotrusions, or voids of the film. This figure shows that a substantial number of particles or voids may have an x, y, and/or z dimension in the range of about 0.05 μm to about 10 μm.

FIG. 48 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, and at least one substantially right angle. Although not exhaustive, the following descriptions may apply to at least one of the protrusions or particles in this figure when viewed in the yz plane: substantially linear, and substantially rectangular. Although not exhaustive, the following other descriptions may also apply to at least one of the protrusions or particles in this figure: nanoflake, ribbon-shaped, and pseudoplanar.

A scale bar of 1 μm is indicated in the SEM of FIG. 48, which may provide an indication of the size of the nanoparticles, nanoprotrusions, or voids of the film. This figure shows that a substantial number of particles or voids may have an x, y, and/or z dimension in the range of about 0.01 μm to about 5 μm.

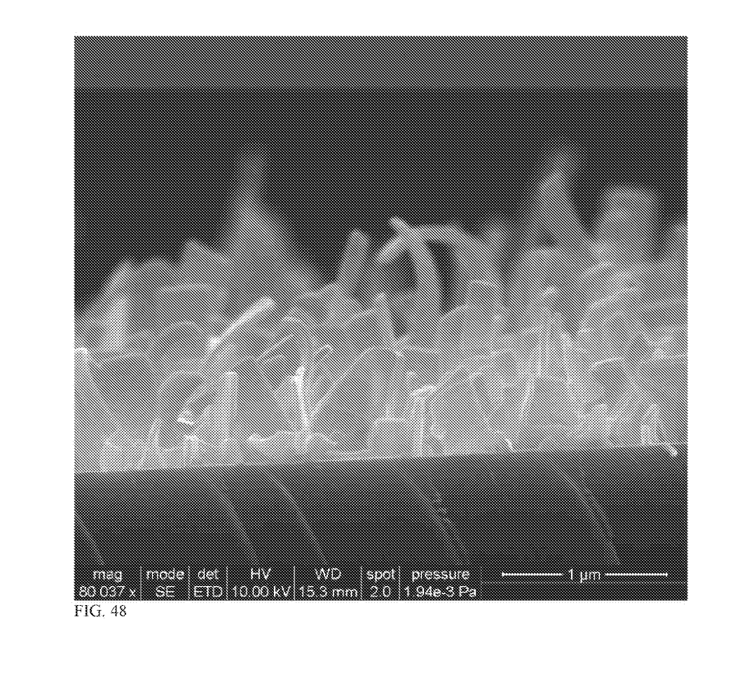

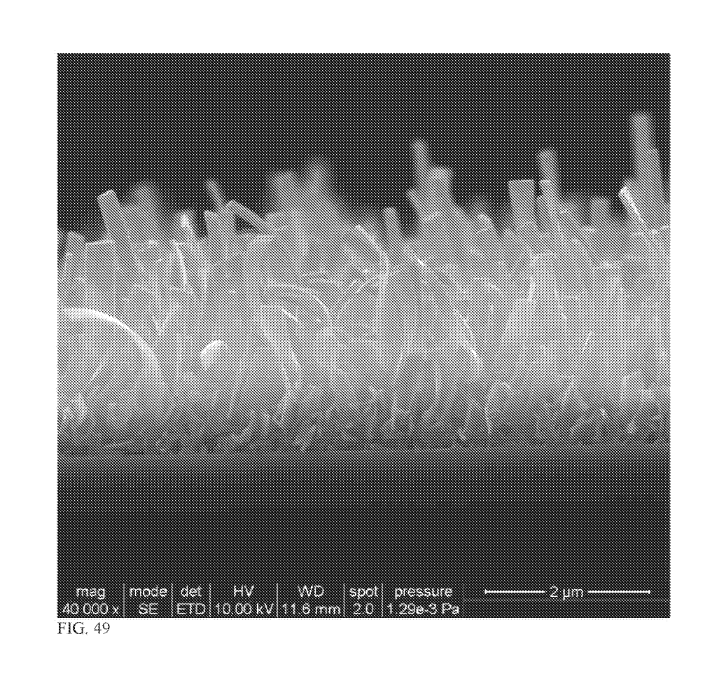

FIG. 49 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, and at least one substantially right angle. Although not exhaustive, the following descriptions may apply to at least one of the protrusions or particles in this figure when viewed in the yz plane: substantially linear, and substantially rectangular. Although not exhaustive, the following other descriptions may also apply to at least one of the protrusions or particles in this figure: nanoflake, ribbon-shaped, and pseudoplanar.

A scale bar of 2 μm is indicated in the SEM of FIG. 49, which may provide an indication of the size of the nanoparticles, nanoprotrusions, or voids of the film. This figure shows that a substantial number of particles or voids may have an x, y, and/or z dimension in the range of about 0.02 μm to about 10 μm.

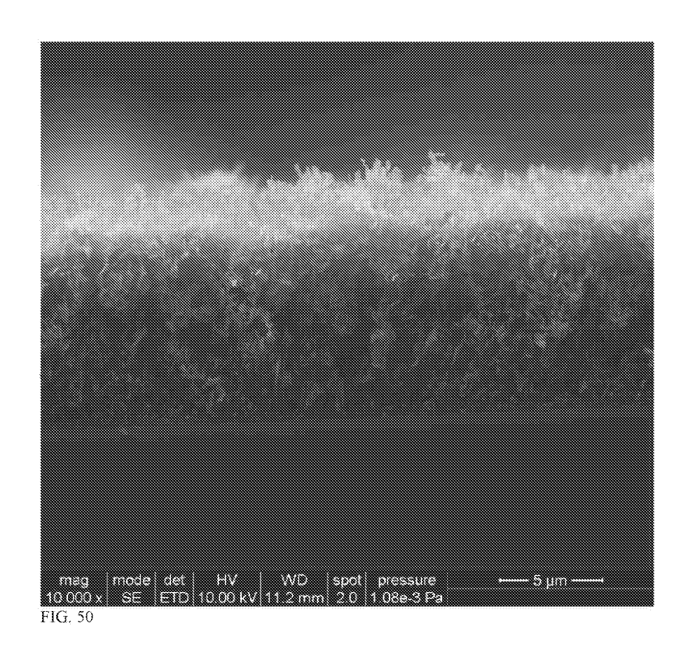

FIG. 50 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: fiber-shaped and ribbon shaped.

A scale bar of 5 μm is indicated in the SEM of FIG. 50, which may provide an indication of the size of the nanoparticles, nanoprotrusions, or voids of the film. This figure shows that a substantial number of particles or voids may have an x, y, and/or z dimension in the range of about 0.01 μm to about 20 μm.

FIG. 51 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: granular, capsule-shaped, fiber-shaped, ribbon-shape, and rod-shaped.

A scale bar of 3 μm is indicated in the SEM of FIG. 51, which may provide an indication of the size of the nanoparticles, nanoprotrusions, or voids of the film. This figure shows that a substantial number of particles or voids may have an x, y, and/or z dimension in the range of about 0.01 μm to about 5 μm.

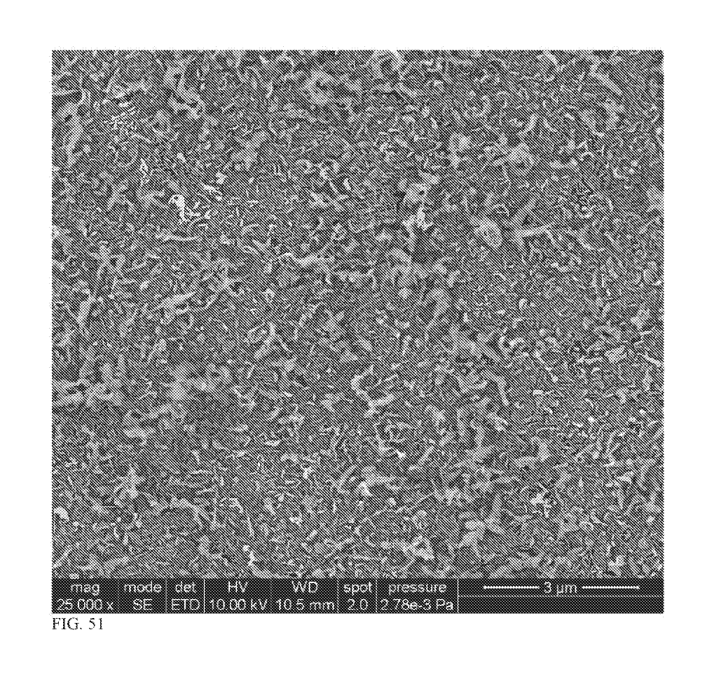

FIG. 52 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: nanoflake, pseudoplanar, ribbon-shaped, and granular.

A scale bar of 4 μm is indicated in the SEM of FIG. 52, which may provide an indication of the size of the nanoparticles, nanoprotrusions, or voids of the film. This figure shows that a substantial number of particles or voids may have an x, y, and/or z dimension in the range of about 0.05 μm to about 10 μm.

FIG. 53 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure: nanoflake, pseudoplanar, ribbon-shaped, and granular.

A scale bar of 3 μm is indicated in the SEM of FIG. 53, which may provide an indication of the size of the nanoparticles, nanoprotrusions, or voids of the film. This figure shows that a substantial number of particles or voids may have an x, y, and/or z dimension in the range of about 0.05 μm to about 10 μm.

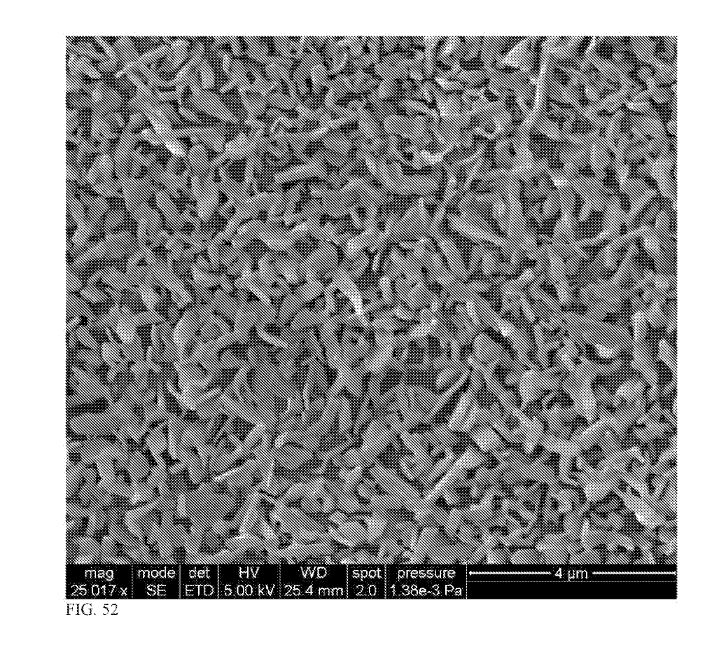

FIG. 54 also depicts an SEM image of a surface of a porous film. This image can illustrate how the terminology described herein may be applied to the structure of protrusions or particles in a porous film. Although not exhaustive, the following descriptions may apply to at least one of the protrusions or particles in this figure when viewed in the xy plane: substantially rectangular, at least one substantially right angle, and substantially all substantially right angles. Although not exhaustive, the following descriptions may apply to at least one of the protrusions or particles in this figure when viewed in the yz plane: substantially linear, substantially rectangular, at least one substantially right angle, and substantially all substantially right angles. Although not exhaustive, the following other descriptions may also apply to at least one of the protrusions or particles in this figure: nanoflake, ribbon-shaped, pseudoplanar.

A scale bar of 400 nm is indicated in the SEM of FIG. 54, which may provide an indication of the size of the nanoparticles, nanoprotrusions, or voids of the film. This figure shows that a substantial number of particles or voids may have an x, y, and/or z dimension in the range of about 50 nm to about 2000 nm.

Various shapes and dimensions are recited herein with respect to several examples of images and figures of associated with various examples of porous films. These shapes and dimensions are provided merely to help provide an understanding of the terminology used, and are not intended to be exhaustive descriptions for any particular example or figure. Thus, the omission of any particular term with respect to any particular example or figure does not suggest that the particular term does not apply to the particular example or figure.

In some embodiments, an angle between the plane of the individual nanostructures and the film may be any value between 0 and 90 degrees with equal probability and/or it may be that no particular angle is preferred. In other words, it may be that no particular general alignment or substantial orientation is exhibited by the nanostructures of this film.

The thickness of a porous film may vary. In some embodiments, a porous film may have a thickness in the nanometer to μm range. For example, the thickness of the film may be about 500 nm to about 100 μm, about 1 μm to about 20 μm, about 1 μm to about 10 μm, about 1 μm to about 5 μm, about 500 nm, about 0.1 μm, about 1 μm, about 1.3 μm, about 3 μm, or about 4 μm, about 5 μm, about 7 μm, about 10 μm, about 20 μm, about 100 μm, or any thickness in a range bounded by, or between, any of these values.

A porous film may comprise a number of pores or voids. For example, a porous film may comprises a plurality of voids having a total volume that may be about 50%, about 70%, about 80%; about 85%, about 90%, about 95%, or about 99% of the volume of the film, or any percentage of total volume in a range bounded by, or between, any of these values.

In some embodiments, a film may comprises a plurality of voids of a number and size such that the film may have a thickness that is about 2 times, about 10 times; up to about 50 times, or 100 times, that of the thickness of a film of the same material which has no voids, or any thickness ratio in a range bounded by, or between, any of these values. For example, a film may have a thickness of about 5 μm when a film of the same material would have a thickness of 800 nm if the film had no voids.

The size of the voids may vary. The dimensions of a void may be quantified as described above for a particle or protrusion. In some embodiments, at least about 10% of the voids have a largest dimension, or an x dimension, of about 0.5 μm to about 5 μm, about 1 μm to about 5 μm, about 0.5 μm, about 1 μm, about 2 μm, about 3 μm, about 4 μm, about 5 μm, or any length in a range bounded by, or between, any of these values. In some embodiments, at least one void in the film, or average of the voids in the film, may have an x dimension, a y dimension, or a z dimension of: about 5 nm to about 1000 μm, about 1 μm to about 1000 μm, about 5 nm, about 0.01 μm, about 0.02 μm, about 0.05 μm, about 0.1 μm, about 0.5 μm, about 1 μm, about 2 μm, about 5 μm, about 10 μm, about 20 μm, about 50 μm, about 100 μm, about 150 μm, about 200 μm, about 500 μm, about 1000 μm, or any length in a range bounded by, or between, any of these values.

The density of a porous film may vary, and may be affected by the voids, the material, and other factors. In some embodiments, the density of the film including the voids may be about 0.005 picograms/μm³, about 0.05 picograms/μm³, about 0.1 picograms/μm³, about 0.3 picograms/μm³, 0.5 picograms/μm³, about 0.7 picograms/μm³, about 0.9 picograms/μm³, or any density in a range bounded by, or between, any of these values.

The refractive index of the material of the porous film may vary. For example, the refractive index may be about 1.1, about 1.5, about 1.7, about 1.8, or any refractive index in a range bounded by, or between, any of these values. In some embodiments, the refractive index of the material of the porous film may be greater than or equal to that of the substrate.

A porous film may comprise a material that includes an organic compound, such as a non-polymeric organic compound that may comprise an optionally substituted aromatic ring. In some embodiments, the porous film may comprise at least one of the compounds below:

In some embodiments, a porous film may comprise an organic compound, including a nonpolymeric organic compound with a molecular weight in the range of about 60 g/mol to about 2000 g/mol or about 120 g/mol to about 1000 g/mol.

Some porous films comprise an optionally substituted aromatic or heteroaromatic ring or ring system, such as optionally substituted phenyl, optionally substituted pyridinyl, optionally substituted carbazolyl, optionally substituted benzimidazole, optionally substituted benzoxazole, optionally substituted benzothiazole, etc.

In some embodiments, the porous film may comprise a linear compound, such as materials wherein non-terminal rings are optionally substituted 1,3-phenylene, optionally substituted 1,4-phenylene, optionally substituted 2,4-pyridinylene, optionally substituted 2.5-pyridinylene, or a similarly attached monocyclic arylene. Some porous films may include a compound having a terminal benzothiazole or benzoxazole.

Some porous films comprise optionally substituted 4-(benzoxazol-2-yl)-4′-(4-diphenylaminophenyl)-3,3′-bipyridine; optionally substituted 4-(benzoxazol-2-yl)-4″-(carbazol-1-yl)terphenyl; optionally substituted 2-(4″-(9H-carbazol-9-yl)-[1,1′:4′,1″-terphenyl]-4-yl)benzo[d]thiazole; optionally substituted 4-(benzoxazol-2-yl)-4″-[di(4-methylphenyl)amino]terphenyl; optionally substituted 4″-(benzothiazol-2-yl)-4″-[di(4-methylphenyl)amino]terphenyl; optionally substituted 4″-(benzo[d]oxazol-2-yl)-N,N-diphenyl-[1,1′:4′,1″-terphenyl]-4-amine; optionally substituted 5,5′-bis(benzoxazol-2-yl)-3,3′-bipyridine; optionally substituted 5,5′-bis(benzothiazol-2-yl)-3,3′-bipyridine; 3,3′-bis(benzoxazol-2-yl)-2,2′-bipyridine; optionally substituted 3,3′-bis(benzo[d]thiazol-2-yl)-2,2′-bipyridine; optionally substituted 5,5′-bis(1-phenyl-1H-benzo[d]imidazol-2-yl)-3,3′-bipyridine optionally substituted; optionally substituted 3,5-di[3-(benzoxazol-2-yl)phenyl]pyridine (IOC-1); optionally substituted 3,5-bis(3-(benzo[d]thiazol-2-yl)phenyl)pyridine; optionally substituted 3,5-di[5-(benzoxazol-2-yl)pyridin-3-yl]benzene; optionally substituted 1,3-bis(5-(benzo[d]thiazol-2-yl)pyridin-3-yl)benzene; optionally substituted 5,5″-bis(benzoxazol-2-yl)-3,3′:5′,3″-terpyridine (IOC-2); optionally substituted 5,5″-bis(benzothiazol-2-yl)-3,3′:5′,3″-terpyridine; optionally substituted 4-(benzoxazol-2-yl)-4″-[di(4-methylphenyl)amino]terphenyl; optionally substituted 4-(benzoxazol-2-yl)-4″-(diphenylamino)terphenyl; optionally substituted 4-(benzothiazol-2-yl)-4″-(diphenylamino)terphenyl; optionally substituted 4-(benzothiazol-2-yl)-4′-(4-diphenylaminophenyl)-3,3′-bipyridine; optionally substituted 4-(benzothiazol-2-yl)-4′4-[4-(carbazol-1-yl)phenyl]-3,3′-bipyridine; optionally substituted 4-(benzoxazol-2-yl)-4′-[4-(carbazol-1-yl)phenyl]-3,3′-bipyridine; optionally substituted 6,6′-bis(benzo[d]thiazol-2-yl)-3,3′-bipyridine, optionally substituted 6,6′-bis(benzo[d]oxazol-2-yl)-3,3′-bipyridine; optionally substituted 3,5-di[5-(benzothiazol-2-yl)pyridin-3-yl]-1-methylbenzene; optionally substituted 3,5-di[5-(benzoxazol-2-yl)pyridin-3-yl]-1-methylbenzene; optionally substituted 3,3″-bis(benzo[d]oxazol-2-yl)-1,1′:3′,1″-terphenyl; optionally substituted 2,2′-(5′-vinyl-[1,1′:3′,1″-terphenyl]-3,3″-diyl)bis(benzo[d]oxazole); optionally substituted 3,5-di([1,1′-biphenyl]-3-yl)pyridine; optionally substituted 1,1′:3′,1″:3″,1′″:3′″,1″″-quinquephenyl; optionally substituted 3,3′,5,5′-tetrakis(benzo[d]oxazol-2-yl)-1,1′-biphenyl (IOC-3); or optionally substituted 3,3′,5,5′-tetrakis(benzo[d]thiazol-2-yl)-1,1′-biphenyl.

Unless otherwise indicated, when a compound or chemical structural feature such as aryl is referred to as being “optionally substituted,” it includes a feature that has no substituents (i.e. be unsubstituted), or a feature that is “substituted,” meaning that the feature has one or more substituents. The term “substituent” has the ordinary meaning known to one of ordinary skill in the art, and includes a moiety that replaces one or more hydrogen atoms attached to a parent compound or structural feature. In some embodiments, the substituent may be an ordinary organic moiety known in the art, which may have a molecular weight (e.g. the sum of the atomic masses of the atoms of the substituent) of 15 g/mol to 50 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, the substituent comprises: 0-30, 0-20, 0-10, or 0-5 carbon atoms; and 0-30, 0-20, 0-10, or 0-5 heteroatoms independently selected from: N, O, S, Si, F, Cl, Br, or I; provided that the substituent comprises at least one atom selected from: C, N, O, S, Si, F, Cl, Br, or I. Examples of substituents include, but are not limited to, alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, amino, etc.

For convenience, the term “molecular weight” is used with respect to a moiety or part of a molecule to indicate the sum of the atomic masses of the atoms in the moiety or part of a molecule, even though it may not be a complete molecule.

Structures associated with some of the chemical names referred to herein are depicted below. These structures may be unsubstituted, as shown below, or a substituent may independently be in any position normally occupied by a hydrogen atom when the structure is unsubstituted. Unless a point of attachment is indicated by

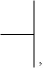

attachment may occur at any position normally occupied by a hydrogen atom.

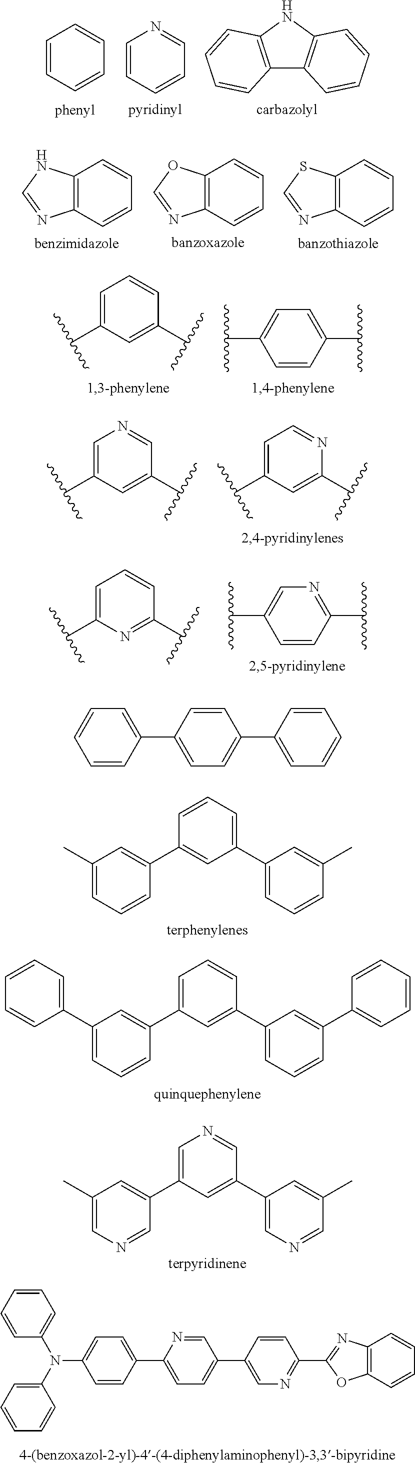

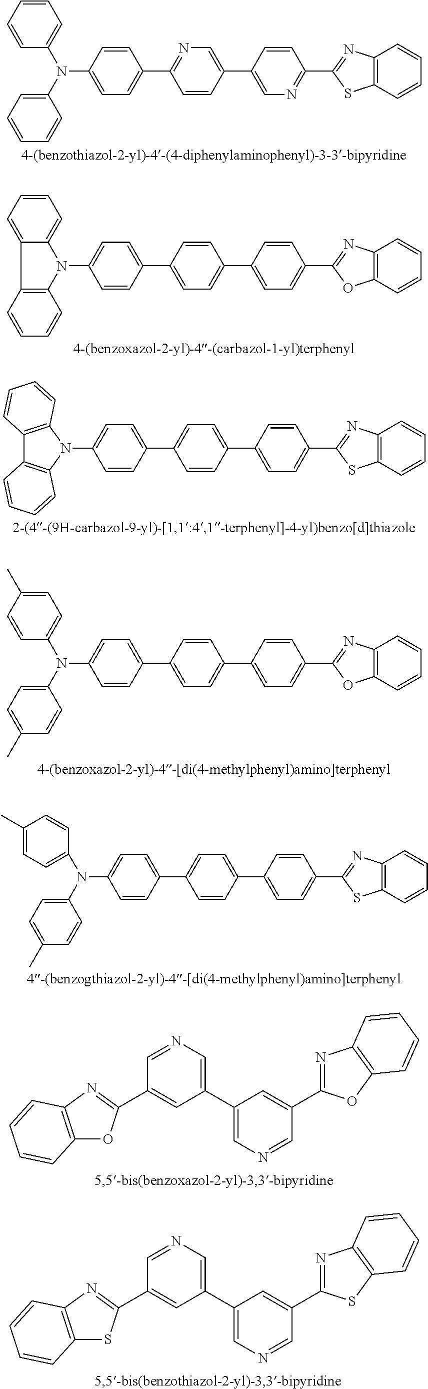

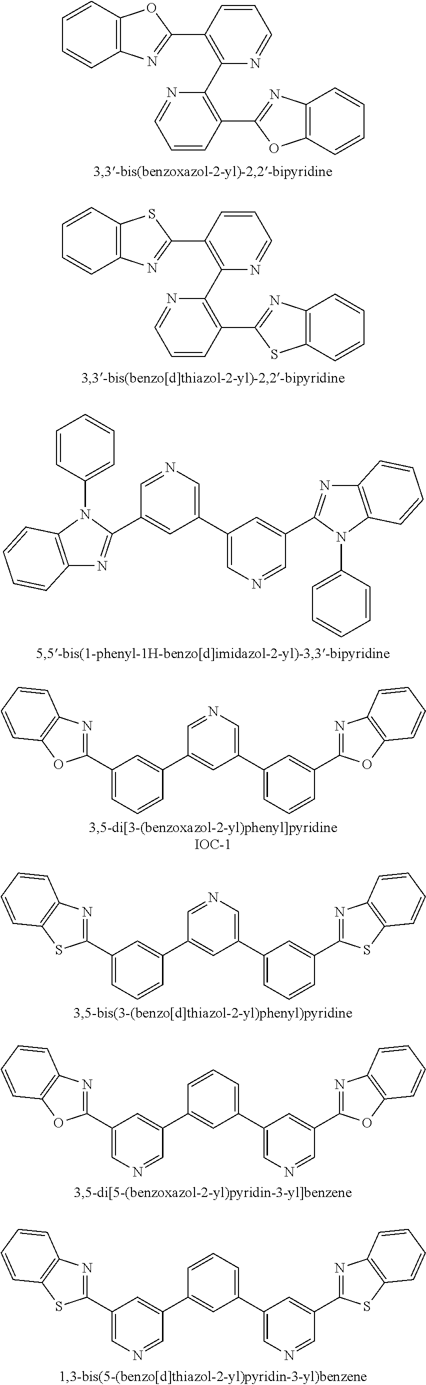

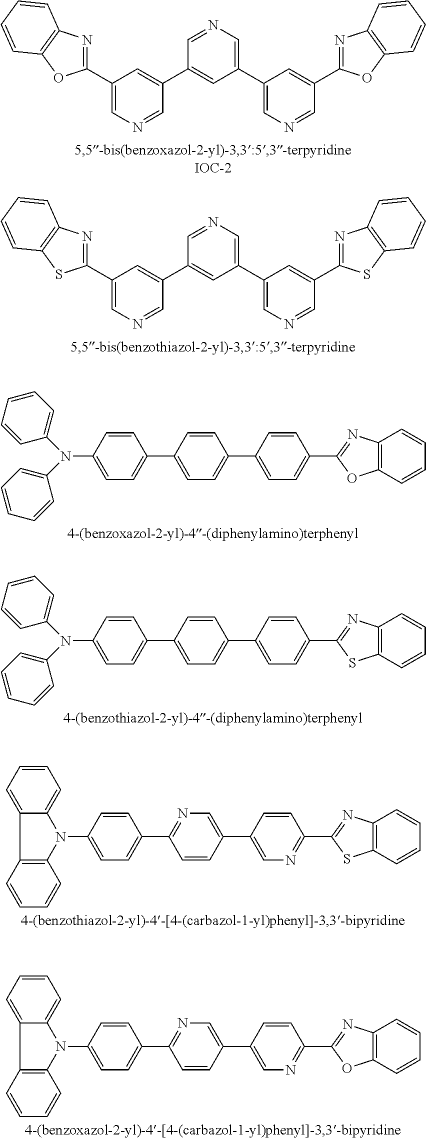 [SMILES: c1ccc2oc(-c3cncc(-c4cncc(-c5cncc(-c6nc7ccccc7o6)c5)c4)c3)nc2c1 ; c1ccc2sc(-c3cncc(-c4cncc(-c5cncc(-c6nc7ccccc7s6)c5)c4)c3)nc2c1 ; c1ccc(N(c2ccccc2)c2ccc(-c3ccc(-c4ccc(-c5nc6ccccc6o5)cc4)cc3)cc2)cc1 ; c1ccc(N(c2ccccc2)c2ccc(-c3ccc(-c4ccc(-c5nc6ccccc6s5)cc4)cc3)cc2)cc1 ; c1ccc2sc(-c3ccc(-c4ccc(-c5ccc(-n6c7ccccc7c7ccccc76)cc5)nc4)cn3)nc2c1 ; c1ccc2oc(-c3ccc(-c4ccc(-c5ccc(-n6c7ccccc7c7ccccc76)cc5)nc4)cn3)nc2c1]

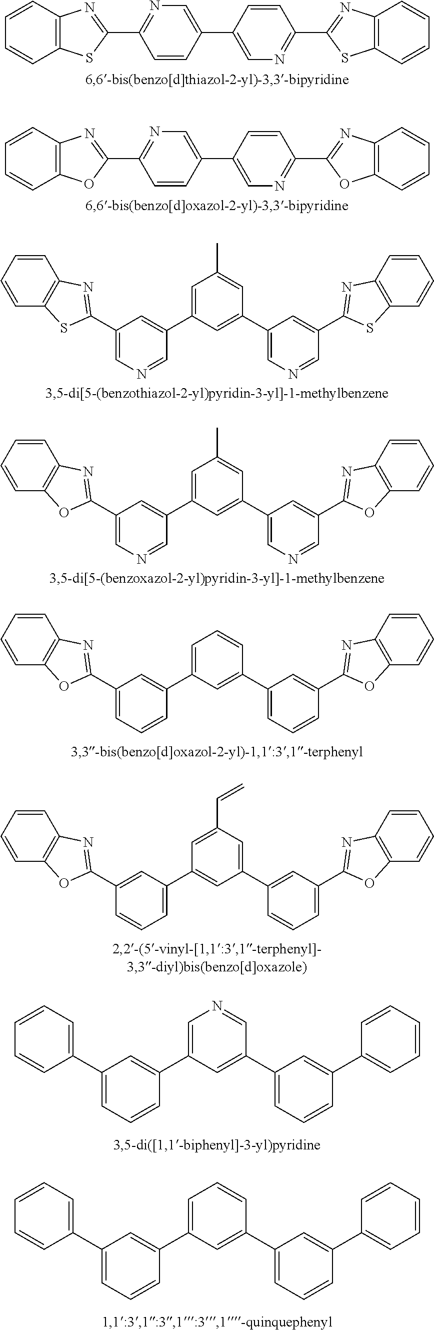

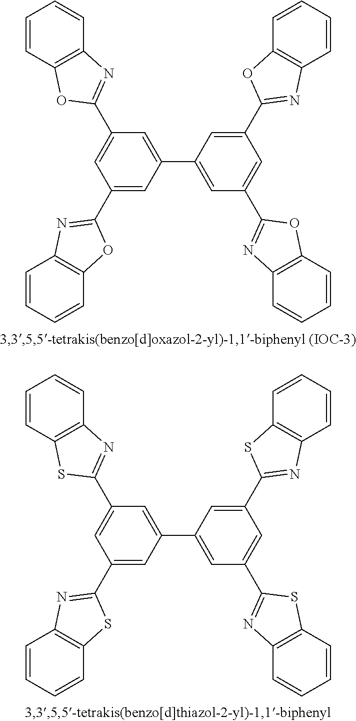 [SMILES: c1ccc2oc(-c3cc(-c4cc(-c5nc6ccccc6o5)cc(-c5nc6ccccc6o5)c4)cc(-c4nc5ccccc5o4)c3)nc2c1 ; c1ccc2sc(-c3cc(-c4cc(-c5nc6ccccc6s5)cc(-c5nc6ccccc6s5)c4)cc(-c4nc5ccccc5s4)c3)nc2c1]

These compounds may be prepared by the methods described in US 2010/0326526, US 2012/0223275, US 2012/0179089, and US 2012/0226046, all of which are Incorporated by reference herein for all disclosure related to these compounds.

In some embodiments, an internal ring, such as optionally substituted 1,3-phenylene, optionally substituted 1,4-phenylene, optionally substituted 2,4-pyridinylene, or optionally substituted 2,5-pyridinylene, may be unsubstituted, or may have substituents with a small steric bulk, such as F, Cl, OH, NH₂, CN, etc. In some embodiments, terminal rings may be unsubstituted, or may have substituents such as R′, —OR′, —COR′, —CO₂R′, —OCOR′, —NR′COR″, CONR′R″, —NR′R″, F; CI; Br; I; nitro; CN, etc., wherein R′ and R″ are independently H, optionally substituted phenyl, or C₁₋₆ alkyl, such as methyl, ethyl, propyl isomers, cyclopropyl, butyl isomers, cyclobutyl isomers (such as cyclobutyl, methylcyclopropyl, etc.), pentyl isomers, cyclopentyl isomers, hexyl isomers, cyclohexyl isomers, etc.

Other compounds that may be useful in porous films include any compound described in one of the following documents: U.S. Provisional Application No. 61/449,034, filed Mar. 3, 2011, which is incorporated by reference for all disclosure related to the preparation, structure, and use of chemical compounds; U.S. Provisional Application No. 61/221,472, filed Jun. 29, 2009, which is incorporated by reference for all disclosure related to the preparation, structure, and use of chemical compounds; US 2010/0326526 (U.S. patent application Ser. No. 12/825,953, filed Jun. 29, 2010), which is incorporated by reference for all disclosure related to the preparation, structure, and use of chemical compounds; U.S. Provisional Patent Application No. 61/383,602, filed Sep. 16, 2010, which is incorporated by reference for all disclosure related to the preparation, structure, and use of chemical compounds; U.S. Provisional Application No. 61/426,259, filed Dec. 22, 2010; which is incorporated by reference for all disclosure related to the preparation, structure, and use of chemical compounds; and copending applications US 2012/0179089 (Ser. No. 12/232,837, filed Sep. 14, 2011), which is incorporated by reference for all disclosure related to the preparation, structure, and use of chemical compounds; US 2012/0223275 (Ser. No. 13/410,602, filed Mar. 2, 2012), which is incorporated by reference for all disclosure related to the preparation, structure, and use of chemical compounds; US 2012/0226046 (Ser. No. 13/410,778, filed Mar. 2, 2012), which is incorporated by reference for all disclosure related to the preparation, structure, and use of chemical compounds; US 2011/0140093 Ser. No. 13/033,473, filed Feb. 23, 2011, which is incorporated by reference for all disclosure related to the preparation, structure, and use of chemical compounds; and Ser. No. 61/696,035, filed Aug. 31, 2012, which is incorporated by reference for all disclosure related to the preparation, structure, and use of chemical compounds.

A porous film may be prepared by depositing a material on a surface, such as a substrate. For example, the deposition may be vapor deposition, which may be carried out under high temperature and/or high vacuum conditions; or the porous film may be deposited by drop casting or spin casting. In some embodiments, the material may be deposited on a substantially transparent substrate. Deposition and/or annealing conditions may affect the characteristics of the film.

The rate of deposition of the material on a surface may vary. For example, the deposition rate may be: about 0.1 A/sec, about 0.2 A/sec, about 1 A/sec, about 10 A/sec, about 100 A/sec, about 500 A/sec, about 1000 Å/sec, or any value in a range bounded by, or between, any of these deposition rates.

The material may be deposited onto a variety of surfaces to form a film. For some devices, the material may be deposited onto an anode, a cathode, or a transparent layer.

A material that has been deposited on a surface may be further treated by heating or annealing. The temperature of heating may vary. For example, the a precursor material may be heated at a temperature of about 80° C., about 100° C., about 110° C., about 120° C., about 150° C., about 180° C., about 200° C., about 130° C., about 260° C., about 290° C., or any temperature in a range bounded by, or between, any of these values.

The time of heating may also vary. For example, the material may be heated for about 5 minutes, about 15 minutes, about 30 minutes, about 60 minutes, about 2 hours, about 5 hours, about 10 hours, about 20 hours, or any amount of time in a range bounded by, or between, any of these values. In some embodiments, a material may be heated at about 100° C. to about 260° C. for about 5 minutes to about 30 minutes.

Generally, a porous film may be deposited on at least part of a surface of a layer in a device to provide an outcoupling or a scattering effect. For outcoupling, a porous film may deposited on at least part of a surface of any internally reflective layer, including any layer that may reflect light instead of allowing it to pass to an adjacent layer, such as an emissive layer, an anode, a cathode, any transparent layer, etc. In some embodiments, a transparent layer may be disposed between the anode and the film, the cathode and the film, etc.

A light-emitting device comprising a porous film may have a variety of configurations. For example, a light emitting device may include an anode, a cathode and an emissive layer disposed between the anode and cathode.

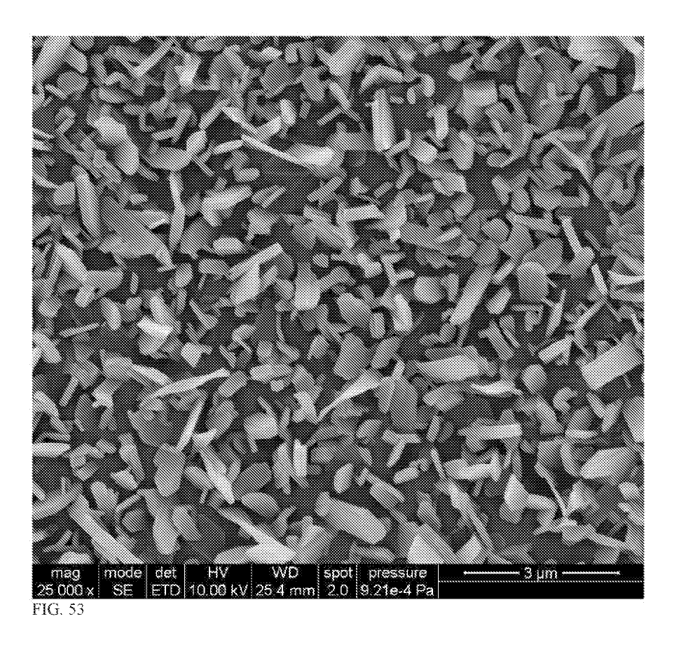

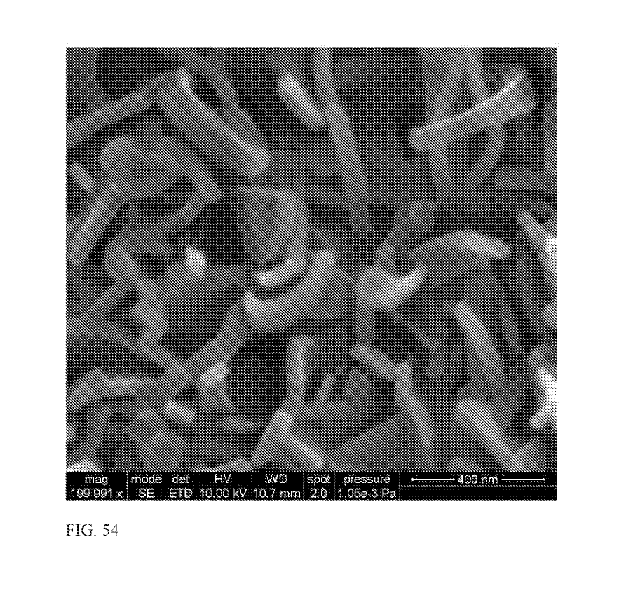

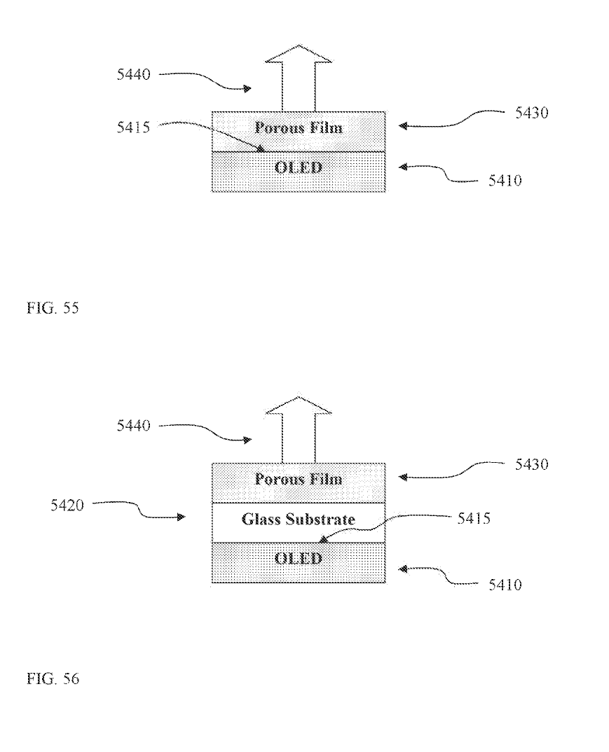

With reference to FIGS. 55 and 56, a porous film 5430 may be disposed over the emitting surface 5415 of an OLED 5410. In some embodiments, the porous film 5430 is disposed directly on the emitting surface 5415 of an OLED 5410 (FIG. 55) and functions as an outcoupling film. Emitted light 5440 from the OLED 5410 may pass through the porous film 5430. In some embodiments, a glass substrate 5420 (FIG. 56) may be disposed between the OLED 5410 and the porous film 5430, wherein the glass substrate 5420 is in contact with or adjacent to the light emitting surface 5415 of the OLED 5410. Emitted light 5440 may pass from the OLED 5410 through the glass substrate 5420 and out of the porous film 5430. The porous film 5430 functions as an outcoupling film.

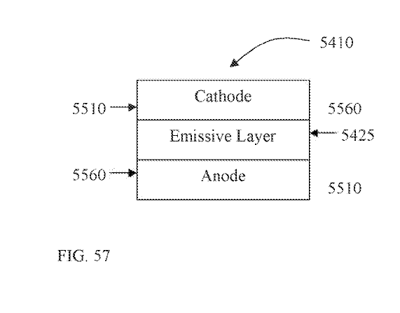

Some examples of OLEDs 5410 that may be suitable for the devices depicted in FIGS. 55-56 are depicted in FIG. 57. Generally, an emissive layer 5425 may be disposed between an anode 5560 and a cathode 5510. Other layers, such as an electron-transport layer, a hole-transport layer, an electron-injection layer, a hole-injection layer, an electron-blocking layer, a hole-blocking layer, additional emissive layers, etc., may be present between the emissive layer 5425, and the anode 5560 and/or the cathode 5510. Light may be emitted through the anode 5560 or the cathode 5510.

An anode may be a layer comprising a conventional material such as a metal, a mixed metal, an alloy, a metal oxide or a mixed-metal oxide, a conductive polymer, and/or an inorganic material such as carbon nanotube (CNT). Examples of suitable metals include the Group 1 metals, the metals in Groups 4, 5, 6, and the Group 8-10 transition metals. If the anode layer is to be light-transmitting, metals in Group 10 and 11, such as Au, Pt, and Ag, or alloys thereof; or mixed-metal oxides of Group 12, 13, and 14 metals, such as indium-tin-oxide (ITO), indium-zinc-oxide (IZO), and the like, may be used. In some embodiments, the anode layer may be an organic material such as polyaniline. The use of polyaniline is described in “Flexible light-emitting diodes made from soluble conducting polymer,” Nature, vol. 357, pp. 477-479 (11 Jun. 1992). Examples of suitable high work function metals and metal oxides include but are not limited to Au, Pt, or alloys thereof; ITO; IZO; and the like. In some embodiments, the anode layer can have a thickness in the range of about 1 nm to about 1000 nm.

A cathode may be a layer including a material having a lower work function than the anode layer. Examples of suitable materials for the cathode layer include those selected from alkali metals of Group 1, Group 2 metals, Group 12 metals including rare earth elements, lanthanides and actinides, materials such as aluminum, indium, calcium, barium, samarium and magnesium, and combinations thereof. Li-containing organometallic compounds, LiF, and Li₂O may also be deposited between the organic layer and the cathode layer to lower the operating voltage. In some embodiments a cathode may comprise Al, Ag, Mg, Ca, Cu, Mg/Ag, LiF/Al, CsF, CsF/Al or alloys thereof. In some embodiments, the cathode layer can have a thickness in the range of about 1 nm to about 1000 nm.

A transparent electrode may include an anode or a cathode through which some light may pass. In some embodiments, a transparent electrode may have a relative transmittance of about 50%, about 80%, about 90%, about 100%, or any transmittance in a range bounded by, or between, any of these values.

An emissive layer may be any layer that can emit light. In some embodiments, an emissive layer may comprise an emissive component, and optionally, a host. The device may be configured so that holes can be transferred from the anode to the emissive layer and/or so that electrons can be transferred from the cathode to the emissive layer. If a host is present, the amount of the host in an emissive layer may vary. For example, the host may be about 50%, about 60%, about 90%, about 97%, or about 99% by weight of the emissive layer, or may be any percentage in a range bounded by, or between, any of these values.

In some embodiments, Compound 10 may be the host in an emissive layer.

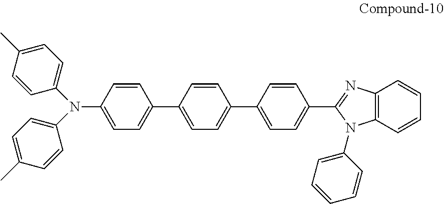 [SMILES: Cc1ccc(N(c2ccc(C)cc2)c2ccc(-c3ccc(-c4ccc(-c5nc6ccccc6n5-c5ccccc5)cc4)cc3)cc2)cc1]

The amount of an emissive component in an emissive layer may vary. For example, the emissive component may be about 0.1%, about 1%, about 3%, about 5%, about 10%, or about 100% of the weight of the emissive layer, or may be any percentage in a range bounded by, or between, any of these values. In some embodiments, the emissive layer may be a neat emissive layer, meaning that the emissive component is about 100% by weight of the emissive layer, or alternatively, the emissive layer consists essentially of emissive component.

The emissive component may be a fluorescent and/or a phosphorescent compound. In some embodiments, the emissive component comprises a phosphorescent material. Some non-limiting examples of emissive compounds may include: PO-01, bis-{2-[3,5-bis(trifluoromethyl)phenyl]pyridinato-N,C2′}iridium(III)-picolinate, bis(2-[4,6-difluorophenyl]pyridinato-N,C2′)iridium (III) picolinate, bis(2-[4,6-difluorophenyl]pyridinato-N,C2′)iridium(acetylacetonate), Iridium (III) bis(4,6-difluorophenylpyridinato)-3-(trifluoromethyl)-5-(pyridine-2-yl)-1,2,4-triazolate, Iridium (III) bis(4,6-difluorophenylpyridinato)-5-(pyridine-2-yl)-1H-tetrazolate, bis[2-(4,6-difluorophenyl)pyridinato-N,C^{2′}]iridium(III)tetra(1-pyrazolyl)borate, Bis[2-(2′-benzothienyl)-pyridinato-N,C3′]iridium (III)(acetylacetonate); Bis[(2-phenylquinolyl)-N,C2′]iridium (III) (acetylacetonate); Bis[(1-phenylisoquinolinato-N,C2′)]iridium (III) (acetylacetonate); Bis[(dibenzo[f, h]quinoxalino-N,C2′)iridium (III)(acetylacetonate); Tris(2,5-bis-2′-(9′,9′-dihexylfluorene)pyridine)iridium (III); Tris[1-phenylisoquinolinato-N,C2′]iridium (III); Tris-[2-(2′-benzothienyl)-pyridinato-N,C3]iridium (III); Tris[1-thiophen-2-ylisoquinolinato-N,C3′]iridium (III); and Tris[1-(9,9-dimethyl-9H-fluoren-2-yl)isoquinolinato-(N,C3′)iridium (III)), Bis(2-phenylpyridinato-N,C2′)iridium(III)(acetylacetonate) [Ir(ppy)₂(acac)], Bis(2-(4-tolyl)pyridinato-N,C2′)iridium(III)(acetylacetonate) [Ir(mppy)₂(acac)], Bis(2-(4-tert-butyl)pyridnato-N,C2′)iridium (III)(acetylacetonate) [Ir(t-Buppy)₂(acac)], Tris(2-phenylpyridinato-N,C2′)iridium (III) [Ir(ppy)₃], Bis(2-phenyloxazolinato-N,C2′)iridium (III) (acetylacetonate) [Ir(op)₂(acac)], Tris(2-(4-tolyl)pyridinato-N,C2′)iridium(III) [Ir(mppy)₃], Bis[2-phenylbenzothiazolato-N,C2′]iridium (III)(acetylacetonate), Bis[2-(4-tert-butylphenyl)benzothiazolato-N,C2′]iridium(III)(acetylacetonate), Bis[(2-(2′-thienyl)pyridinato-N,C3′)]iridium (III) (acetylacetonate), Tris[2-(9.9-dimethylfluoren-2-yl)pyridinato-(N,C3′)]iridium (III), Tris[2-(9.9-dimethylfluoren-2-yl)pyridinato-(N,C3′)]iridium (III), Bis[5-trifluoromethyl-2-[3-(N-phenylcarbzolyl)pyridinato-N,C2′]iridium(III)(acetylacetonate), (2-PhPyCz)₂Ir(III)(acac), etc.

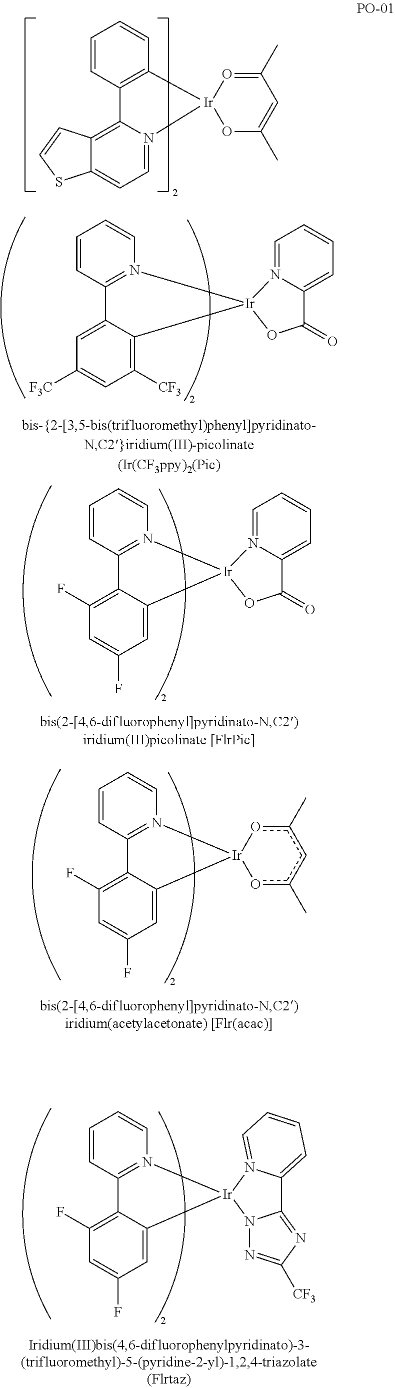

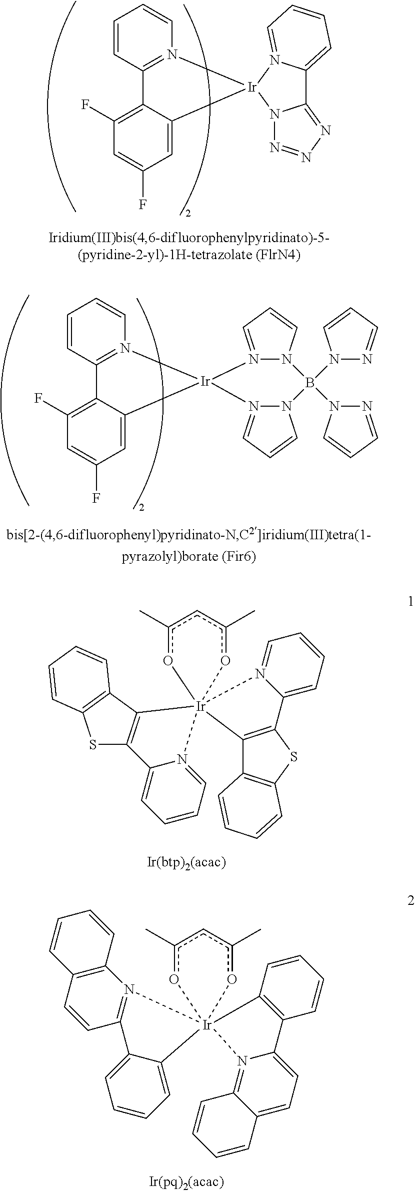

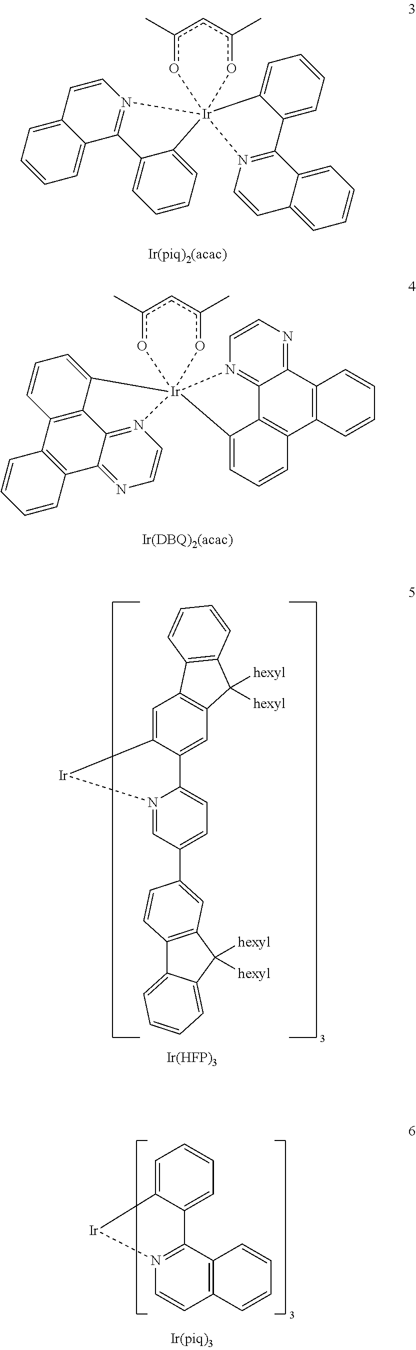

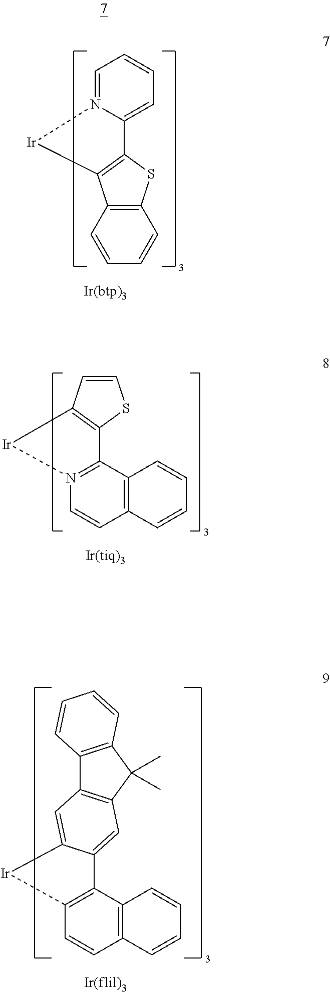

1. (Btp)₂Ir(III)(acac); Bis[2-(2′-benzothienyl)-pyridinato-N,C3′]iridium (III)(acetylacetonate)
2. (Pq)₂Ir(III)(acac); Bis[(2-phenylquinolyl)-N,C2′]iridium (III) (acetylacetonate)
3. (Piq)₂Ir(III)(acac); Bis[(1-phenylisoquinolinato-N,C2′)]iridium (III) (acetylacetonate)
4. (DBQ)₂Ir(acac); Bis[(dibenzo[f, h]quinoxalino-N,C2′)iridium (III)(acetylacetonate)
5. [Ir(HFP)₃], Tris(2,5-bis-2′-(9′,9′-dihexylfluorene)pyridine)iridium (III)
6. Ir(piq)₃; Tris[1-phenylisoquinolinato-N,C2′]iridium (III)
7. Ir(btp)₃, Tris-[2-(2′-benzothienyl)-pyridinato-N,C3′]iridium (III)
8. Ir(tiq)₃, Tris[1-thiophen-2-ylisoquinolinato-N,C3′]iridium (III)
9. Ir(fliq)₃; Tris[1-(9,9-dimethyl-9H-fluoren-2-yl)isoquinolinato-(N,C3′)iridium (III))

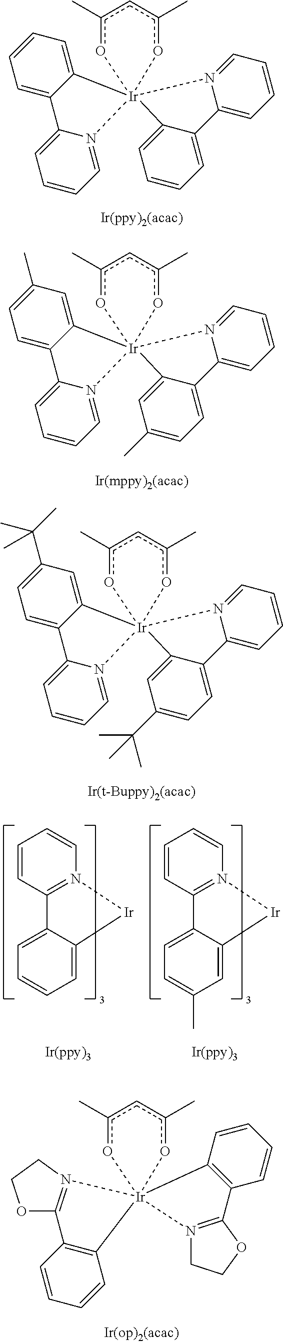

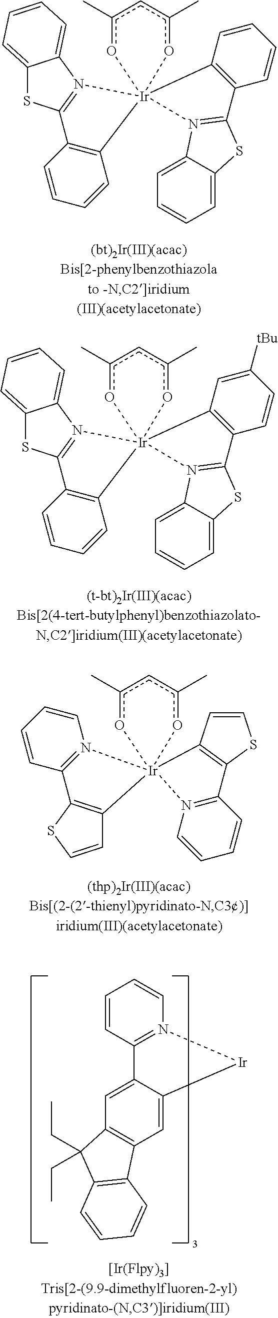

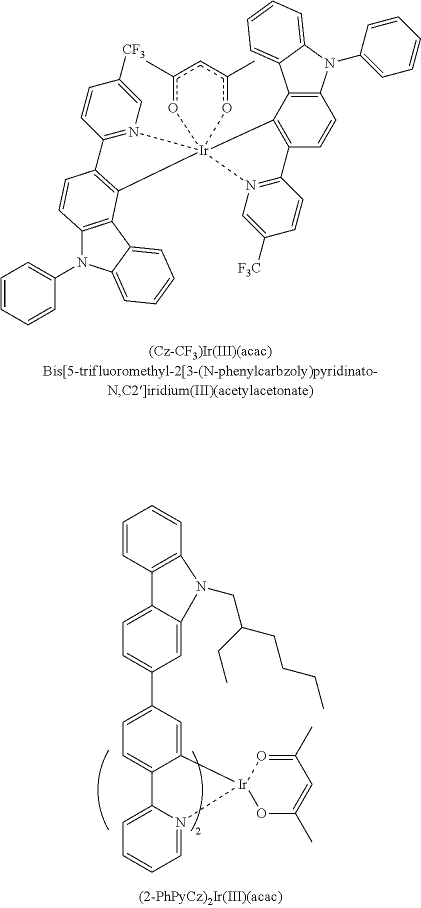

The thickness of an emissive layer may vary. In some embodiments, an emissive layer may have a thickness in the range of about 1 nm to about 150 nm or about 200 nm.

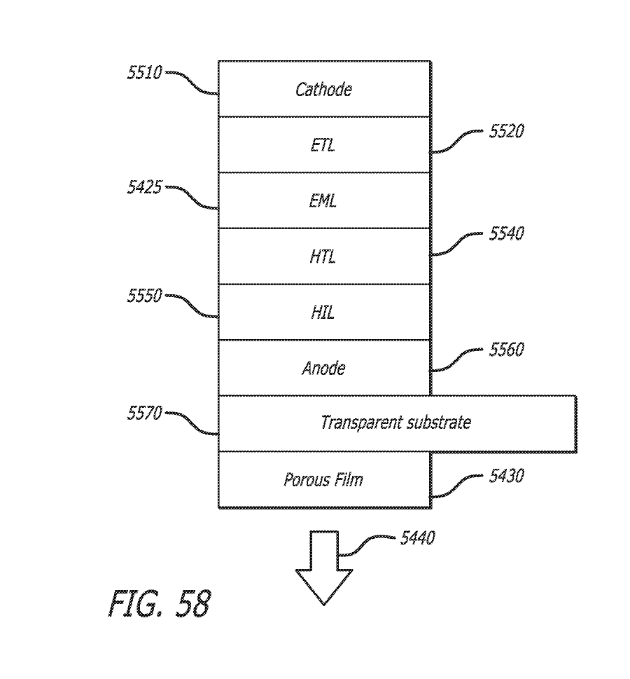

For some OLEDs, additional layers may be present between the emissive layer 5425 and the anode 5560 or between the emissive layer 5425 and the cathode 5510. FIG. 58 depicts an example of a bottom-emitting OLED that may comprise, going in order from bottom to top, a porous film 5430 (bottom), a transparent substrate 5570, an anode 5560, a hole-injection layer 5550, a hole-transport layer 5540, an emissive layer 5425, an electron-transport layer 5520, and a cathode 5510 (top). Each of these layers may contact one another according to the order given above, or additional layers may be present. Light emitted by the emissive layer 5425 may pass through the hole-transport layer 5540, the hole-injection layer 5550, the anode 5560, the transparent substrate 5570, and the porous film 5430 to provide light 5440 emitted by the device through the bottom of the device.

A hole-transport layer may comprise at least one hole-transport material. Examples of hole-transport materials may include: an aromatic-substituted amine, a carbazole, a polyvinylcarbazole (PVK), e.g. poly(9-vinylcarbazole); polyfluorene; a polyfluorene copolymer; poly(9,9-di-n-octylfluorene-alt-benzothiadiazole); poly(paraphenylene); poly[2-(5-cyano-5-methylhexyloxy)-1,4-phenylene]; a benzidine; a phenylenediamine; a phthalocyanine metal complex; a polyacetylene; a polythiophene; a triphenylamine; copper phthalocyanine; 1,1-Bis(4-bis(4-methylphenyl)aminophenyl)cyclohexane; 2,9-Dimethyl-4,7-diphenyl-1,10-phenanthroline; 3,5-Bis(4-tert-butyl-phenyl)-4-phenyl[1,2,4]triazole; 3,4,5-Triphenyl-1,2,3-triazole; 4,4′,4′-tris(3-methylphenylphenylamino)triphenylamine (MTDATA); N,N′-bis(3-methylphenyl)N,N′-diphenyl-[1,1′-biphenyl]-4,4′-diamine (TPD); 4,4′-bis[N-(naphthyl)-N-phenyl-amino]biphenyl (α-NPD); 4,4′,4″-tris(carbazol-9-yl)-triphenylamine (TCTA); 4,4′-bis[N,N′-(3-tolyl)amino]-3,3′-dimethylbiphenyl (HMTPD); 4,4′-N,N′-dicarbazole-biphenyl (CBP); 1,3-N,N-dicarbazole-benzene (mCP); Bis[4-(p,p′-ditolyl-amino)phenyl]diphenylsilane (DTASi); 2,2′-bis(4-carbazolylphenyl)-1,1′-biphenyl (4CzPBP); N,N′N″-1,3,5-tricarbazoloylbenzene (tCP); N,N′-bis(4-butylphenyl)-N,N′-bis(phenyl)benzidine; a combination thereof; or any other material known in the art to be useful as a hole-transport material.

An electron-transport layer may comprise at least one electron-transport material. Examples of electron-transport materials may include: 2-(4-biphenylyl)-5-(4-tert-butylphenyl)-1,3,4-oxadiazole (PBD); 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene; 3-phenyl-4-(1′-naphthyl)-5-phenyl-1,2,4-triazole (TAZ); 2,9-dimethyl-4,7-diphenyl-phenanthroline (bathocuproine or BCP); aluminum tris(8-hydroxyquinolate) (Alq3); and 1,3,5-tris(2-N-phenylbenzimidazolyl)benzene; 1,3-bis[2-(2,2′-bipyridine-6-yl)-1,3,4-oxadiazo-5-yl]benzene (BPY-OXD); 3-phenyl-4-(1′-naphthyl)-5-phenyl-1,2,4-triazole (TAZ), 2,9-dimethyl-4,7-diphenyl-phenanthroline (bathocuproine or BCP); and 1,3,5-tris[2-N-phenylbenzimidazol-z-yl]benzene (TPBI). In some embodiments, the electron transport layer may be aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), a derivative or a combination thereof, or any other material known in the art to be useful as an electron-transport material.

A hole-injection layer may include any material that can inject electrons. Some examples of hole-injection materials may include an optionally substituted compound selected from the following: a polythiophene derivative such as poly(3,4-ethylenedioxythiophene (PEDOT)/polystyrene sulphonic acid (PSS), a benzidine derivative such as N,N,N′,N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), a phthalocyanine metal complex derivative such as phthalocyanine copper (CuPc), a combination thereof, or any other material known in the art to be useful as a hole-injection material. In some embodiments, hole-injection materials, while still being able to transport holes, may have a hole mobility substantially less than the hole mobility of conventional hole-transport materials.

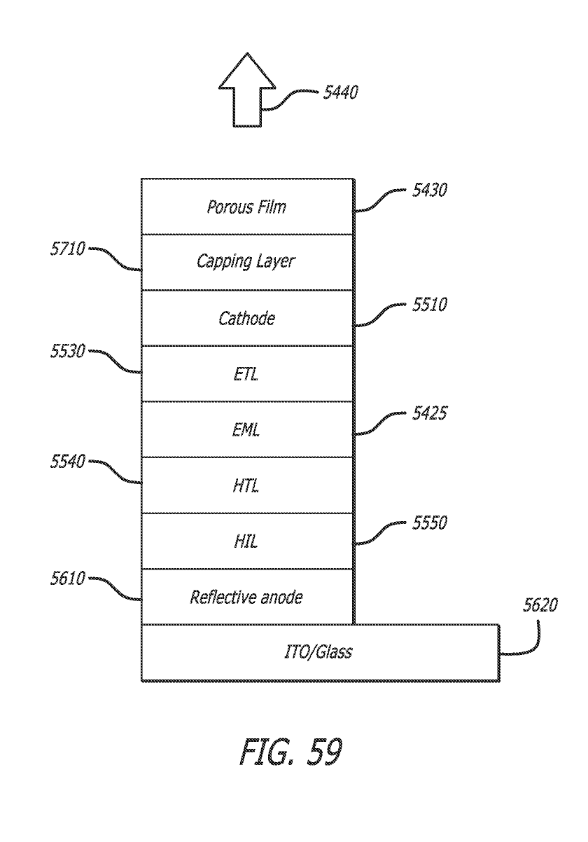

FIG. 59 depicts an example of a top-emitting OLED that may comprise, going from bottom to top, a substrate 5620 (bottom), an anode 5610, such as a reflective anode, a hole-injection layer 5550, a hole-transport layer 5540, an emissive layer 5425, an electron-transport layer 5530, and a cathode 5510, a capping layer 5710, and a porous film 5430 (top). Each of these layers may contact one another according to the order given above, or additional layers may be present. Light that may be emitted by the emissive layer 5425, may pass through the electron-transport layer 5530, the cathode 5510, the capping layer 5710, and the porous film 5430 to provide light 5440 emitted by the device through the top of the device.

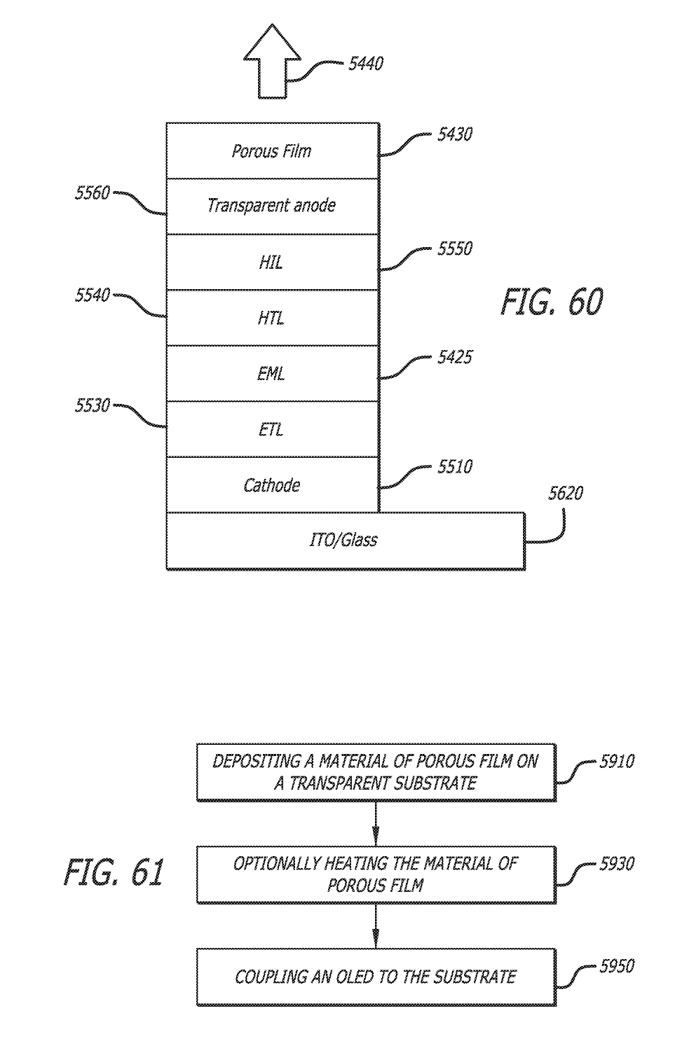

FIG. 60 depicts an example of a top-emitting OLED that may comprise, going from top to bottom, a porous film 5430 (top), an anode 5560, a hole-injection layer 5550, a hole-transport layer 5540, an emissive layer 5425, an electron-transport layer 5530, a cathode 5510, and a substrate 5620 (bottom). Each of these layers may contact one another according to the order given above, or additional layers may be present. Light may be emitted by the emissive layer 5425 and pass through the hole-transport layer 5540, the hole-injection layer 5550, the anode 5560, and the porous film 5430 to provide light 5440 emitted by the top of the device.

A variety of methods may be used to provide a porous film layer to a light-emitting device. FIG. 61 depicts an example of a method that may be used. The first step S910 involves depositing a material of porous film on a transparent substrate. An optional heating step S930 may then be carried out upon the material deposited on the transparent substrate to provide a porous film. Then an OLED is coupled to the substrate using a coupling medium in step S960.

A coupling medium may be any material that has a similar refractive index to the glass substrate and may be capable of causing the glass substrate to be affixed to the OLED, such as by adhesion. Examples may include a refractive index matching oil or double sticky tape. In some embodiments, a glass substrate may have refractive index of about 1.5, and a coupling medium may have refractive index of about 1.4. This may allow light to come through the glass substrate and the coupling medium without light loss.

In some embodiments, the material of the porous film may be deposited directly on the OLED. An optional heating step may also be carried out on the deposited material to provide a porous film. In some embodiments, the heating temperature may be sufficiently low that the performance of the OLED is not adversely affected to a degree that is unacceptable.

A light-emitting device may comprise an encapsulation or protection layer to protect the porous film element from environmental damage, such as damage due to moisture, mechanical deformation, etc. For example, a protective layer may be placed in such a way as to provide a protective barrier between the porous film and the environment.

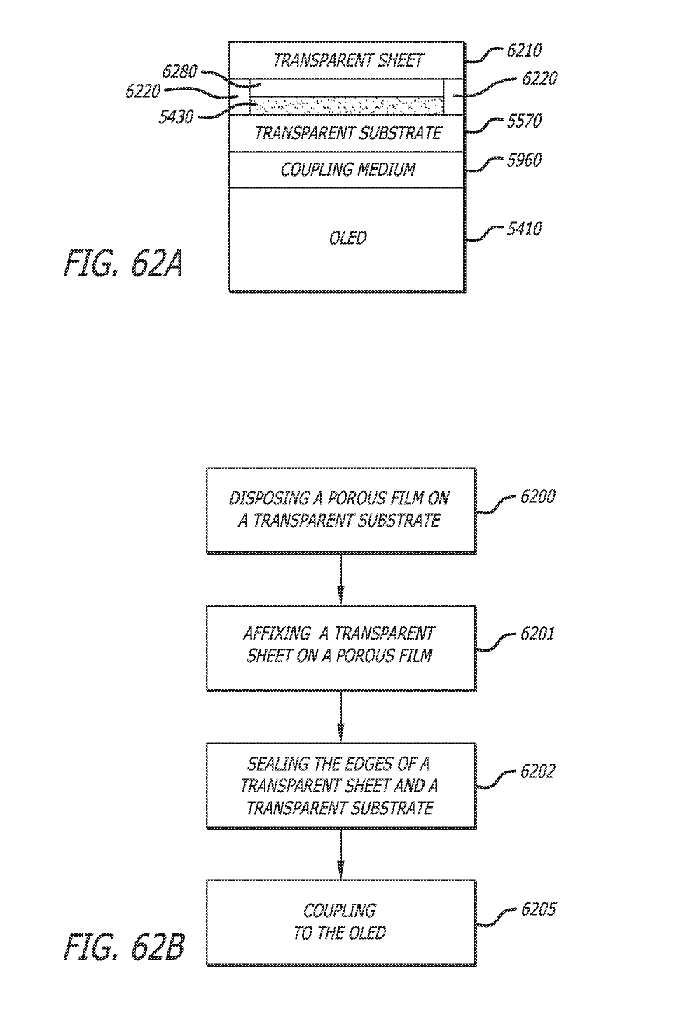

While there may be many ways to encapsulate or protect a porous film, FIG. 62A is a schematic a structure of an encapsulated device and FIG. 62B shows one method that may be used to prepare the device. In this method, step 6200 involves disposing a porous film 5430 on a transparent substrate 5570, and step 6201 involves affixing a transparent sheet 6210 over the porous film 5430. When the transparent sheet 6210 is positioned over the porous film 5430, the edges of the transparent sheet 6210 and the transparent substrate 5570 may be sealed to one another by a sealing material 6220 as shown in step 6202. The sealing material 6220 may be an epoxy resin, a UV-curable epoxy, or another cross-linkable material. Optionally, a gap 6280 may be present between the transparent sheet 6210 and the porous material 5430. A protection layer (i.e., transparent sheet) may also be coated onto the porous film 5430 without sealing the edges of the protection layer 6250 and the transparent substrate 5570. In step 6205, the encapsulated porous film may then be coupled to an OLED 5410 by a coupling medium 5960. If desired, additional layers may be included in the light-emitting device. These additional layers may include an electron injection layer (EIL), a hole-blocking layer (HBL), and/or in exciton blocking layer (EBL).

If desired, additional layers may be included in a light-emitting device. These additional layers may include an electron injection layer (EIL), a hole-blocking layer (HBL), and/or an exciton-blocking layer (EBL).

If present, an electron injection layer may be in a variety of positions in a light-emitting device, such as any position between the cathode layer and the light emitting layer. In some embodiments, the lowest unoccupied molecular orbital (LUMO) energy level of the electron injection material(s) is high enough to prevent it from receiving an electron from the light emitting layer. In other embodiments, the energy difference between the LUMO of the electron injection material(s) and the work function of the cathode layer is small enough to allow the electron injection layer to efficiently inject electrons into the emissive layer from the cathode. A number of suitable electron injection materials are known to those skilled in the art. Examples of suitable electron injection materials may include but are not limited to, an optionally substituted compound selected from the following: LiF, CsF, Cs doped into electron transport material as described above or a derivative or a combination thereof.

If present, a hole-blocking layer may be in a variety of positions in a light-emitting device, such as any position between the cathode and the emissive layer. Various suitable hole-blocking materials that can be included in the hole-blocking layer are known to those skilled in the art. Suitable hole-blocking material(s) include but are not limited to, an optionally substituted compound selected from the following: bathocuproine (BCP), 3,4,5-triphenyl-1,2,4-triazole, 3,5-bis(4-tert-butyl-phenyl)-4-phenyl-[1,2,4]triazole, 2,9-dimethyl-4,7-diphenyl-1,10-phenanthroline, 1,1-bis(4-bis(4-methylphenyl)aminophenyl)-cyclohexane, etc, and combinations thereof.

If present, an exciton-blocking layer may be in a variety of positions in a light-emitting device, such as in any position between the emissive layer and the anode. In some embodiments, the band gap energy of the material(s) that comprise exciton-blocking layer may be large enough to substantially prevent the diffusion of excitons. A number of suitable exciton-blocking materials that can be included in the exciton-blocking layer are known to those skilled in the art. Examples of material(s) that can compose an exciton-blocking layer include an optionally substituted compound selected from the following: aluminum quinolate (Alq₃), 4,4′-bis[N-(naphthyl)-N-phenyl-amino]biphenyl (α-NPD), 4,4′-N,N′-dicarbazole-biphenyl (CBP), and bathocuproine (BCP), and any other material(s) that have a large enough band gap to substantially prevent the diffusion of excitons.

This application incorporates by reference the entire disclosure of US 2012/0223635 (Ser. No. 13/410,812, filed on Mar. 2, 2012).

### Example

### OLED Preparation

An OLED can be prepared as follows. A PEDOT hole injection layer is spin-coated on top of a pre-cleaned ITO/glass, followed by vacuum deposition of the 30 nm-thick α-NPD hole-transport layer at a deposition rate of about 1 Å/s. The emissive layer is added by co-deposition of yellow emitter PO-01 and host Compound-10 at a deposition rate of about 0.05 and about 1 Å/s, respectively, to form an emissive layer having a thickness of about 30 nm. Then TPBI is deposited at about 1 Å/s to a thickness of about 30 nm. LiF is deposited on top of ETL at 0.1 Å/s deposition rate to a thickness of about 1 nm, followed by the deposition of Al at 2 Å/s rate to a thickness of about 100 nm. The base vacuum of the chamber was is about 3×10⁻⁷ torr. A 50-nm thick layer of the compound:

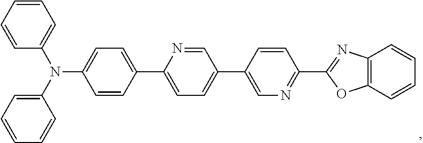 [SMILES: c1ccc(N(c2ccccc2)c2ccc(-c3ccc(-c4ccc(-c5nc6ccccc6o5)nc4)cn3)cc2)cc1]

or another compound suitable for use in a porous film, is deposited on the outer surface of the glass substrate at a deposition rate of about 2 Å/s under a vacuum of about 4×10⁻⁷ torr.

Although the claims have been described in the context of certain preferred embodiments and examples, it will be understood by those skilled in the art that the scope of the claims extend beyond the specifically disclosed embodiments to other alternative embodiments and/or uses and obvious modifications and equivalents thereof.

1. A porous film comprising:

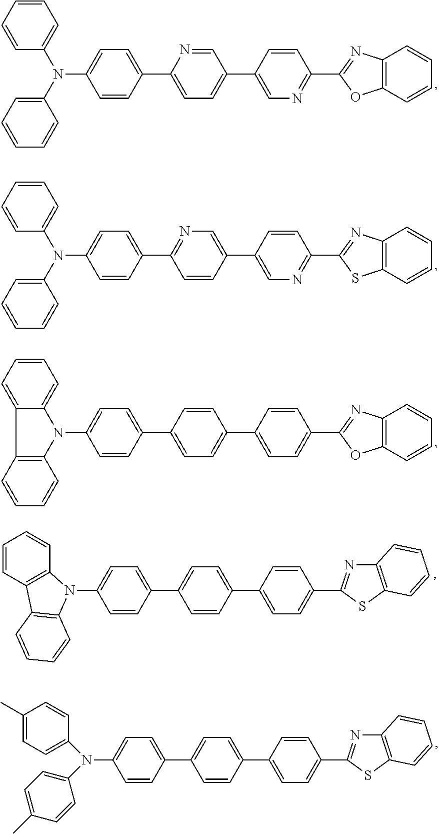

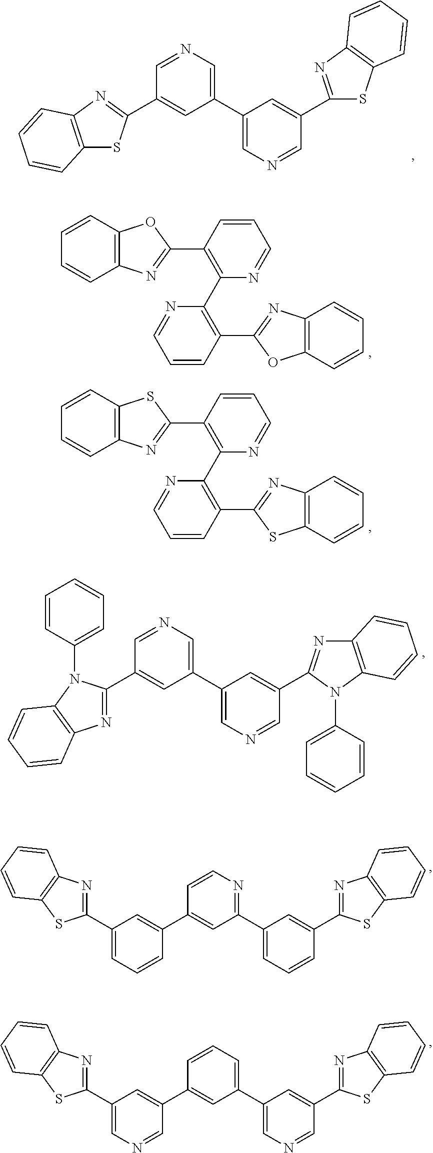

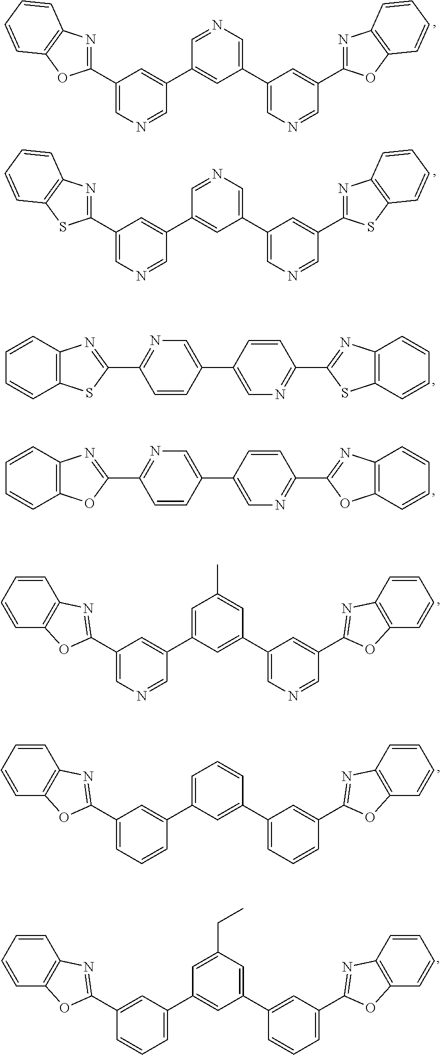

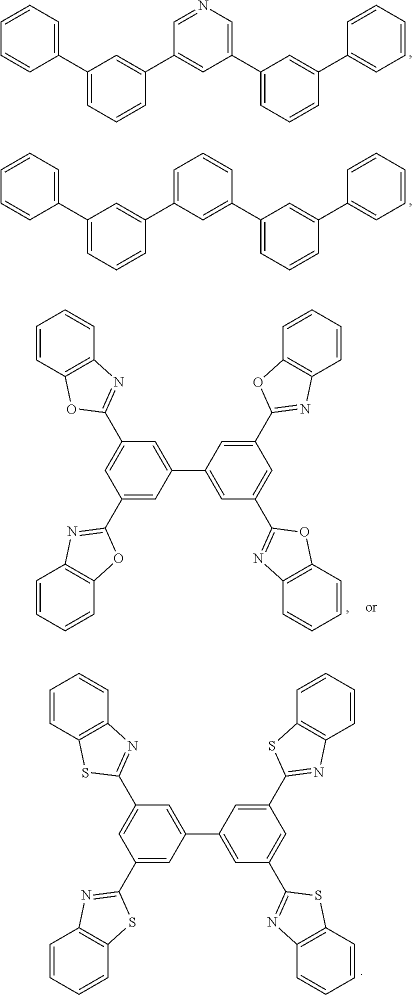

2. The porous film of claim 1, comprising protrusions or particles, having an average x dimension, an average y dimension, or an average z dimension of about 0.1 μm to about 200 μm.

3. The porous film of claim 1, comprising a plurality of voids.

4. The porous film of claim 1, having a thickness of about 0.1 μm to about 20 μm.

5. The porous film of claim 3, having a thickness that is about 2 times to about 100 times the thickness of a film of the same composition having no voids.

6. The porous film of claim 3, wherein the film has a density of about 0.005 pg/μm³ to about 0.9 pg/μm³.

7. The porous film of claim 1, comprising:

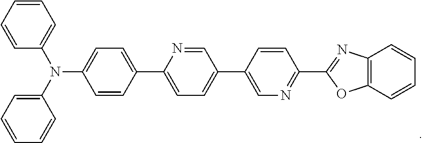 [SMILES: c1ccc(N(c2ccccc2)c2ccc(-c3ccc(-c4ccc(-c5nc6ccccc6o5)nc4)cn3)cc2)cc1]

8. The porous film of claim 1, comprising:

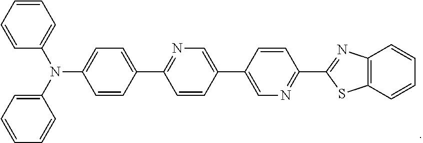 [SMILES: c1ccc(N(c2ccccc2)c2ccc(-c3ccc(-c4ccc(-c5nc6ccccc6s5)nc4)cn3)cc2)cc1]

9. The porous film of claim 1, comprising:

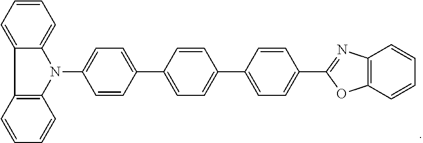 [SMILES: c1ccc2oc(-c3ccc(-c4ccc(-c5ccc(-n6c7ccccc7c7ccccc76)cc5)cc4)cc3)nc2c1]

10. The porous film of claim 1, comprising:

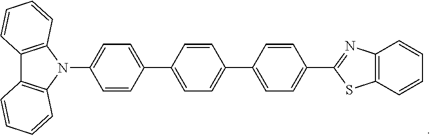 [SMILES: c1ccc2sc(-c3ccc(-c4ccc(-c5ccc(-n6c7ccccc7c7ccccc76)cc5)cc4)cc3)nc2c1]

11. The porous film of claim 1, comprising:

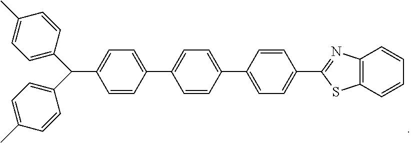 [SMILES: Cc1ccc(C(c2ccc(C)cc2)c2ccc(-c3ccc(-c4ccc(-c5nc6ccccc6s5)cc4)cc3)cc2)cc1]

12. The porous film of claim 1, comprising:

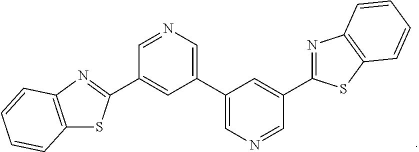 [SMILES: c1ccc2sc(-c3cncc(-c4cncc(-c5nc6ccccc6s5)c4)c3)nc2c1]

13. The porous film of claim 1, comprising:

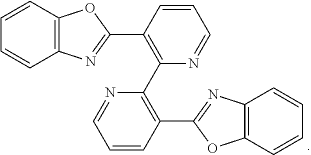 [SMILES: c1cnc(-c2ncccc2-c2nc3ccccc3o2)c(-c2nc3ccccc3o2)c1]

14. The porous film of claim 1, comprising:

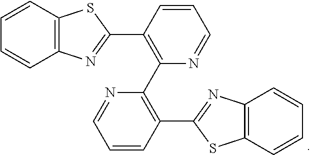 [SMILES: c1cnc(-c2ncccc2-c2nc3ccccc3s2)c(-c2nc3ccccc3s2)c1]

15. The porous film of claim 1, comprising:

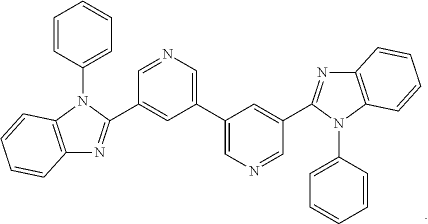 [SMILES: c1ccc(-n2c(-c3cncc(-c4cncc(-c5nc6ccccc6n5-c5ccccc5)c4)c3)nc3ccccc32)cc1]

16. The porous film of claim 1, comprising:

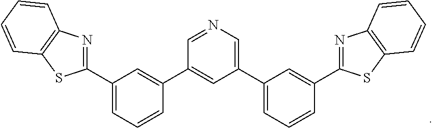 [SMILES: c1cc(-c2cncc(-c3cccc(-c4nc5ccccc5s4)c3)c2)cc(-c2nc3ccccc3s2)c1]

17. The porous film of claim 1, comprising:

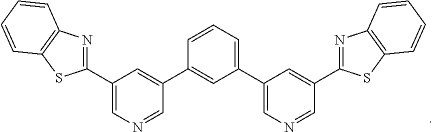 [SMILES: c1cc(-c2cncc(-c3nc4ccccc4s3)c2)cc(-c2cncc(-c3nc4ccccc4s3)c2)c1]

18. The porous film of claim 1, comprising:

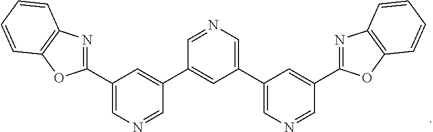 [SMILES: c1ccc2oc(-c3cncc(-c4cncc(-c5cncc(-c6nc7ccccc7o6)c5)c4)c3)nc2c1]

19. The porous film of claim 1, comprising:

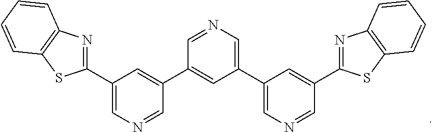 [SMILES: c1ccc2sc(-c3cncc(-c4cncc(-c5cncc(-c6nc7ccccc7s6)c5)c4)c3)nc2c1]

20. The porous film of claim 1, comprising:

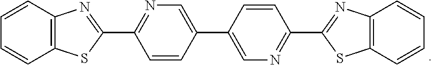 [SMILES: c1ccc2sc(-c3ccc(-c4ccc(-c5nc6ccccc6s5)nc4)cn3)nc2c1]

21-31. (canceled)
